(19) United States
(12) Patent Application Publication
(10) Pub. No.: US 2020/0230066 A1
(43) Pub. Date: Jul. 23, 2020

(54) Self-Assembled Microcapsules for Optically Controlled Cargo Encapsulation and Release

(71) Applicant: The Regents Of The University Of California, Oakland, CA (US)

(72) Inventors: Makiko Tsukamoto Quint, Merced, CA (US); Sayantani Ghosh, Merced, CA (US)

(21) Appl. No.: 16/652,343

(22) PCT Filed: Oct. 8, 2018
(86) PCT No.: PCT/US2018/054871

§ 371 Date: Mar. 30, 2020

Related U.S. Application Data
(60) Provisional application No. 62/569,354, filed on Oct. 6, 2017.

(51) Int. Cl. A61K 9/50; A61K 9/00; A61K 9/51; A61K 47/12

(52) U.S. Cl. CPC A61K 9/5015; A61K 47/12; A61K 9/51; A61K 9/0004

(57) ABSTRACT

Self-assembled organic ligand functionalized microcapsules encapsulating one or more substrates, which release the substrates upon activation with a power source, are provided. Compositions that include these microcapsules, as well as methods of making the microcapsules and releasing the encapsulated substrates are also provided. The structures, compositions and methods find use in a variety of applications, such as drug and cell encapsulation technologies, for direct delivery, control, and activation of medicines and therapies to specific tissues in a living host e.g. targeted cancer therapy and pain management.

### CROSS-REFERENCE

This application claims the benefit of U.S. Provisional Patent Application No. 62/569,354, filed Oct. 6, 2017, which application is incorporated herein by reference in its entirety.

### INTRODUCTION

Robust structures for encapsulating cargo, configured for rapid controlled discharge of its cargo at a particular time and location pose a significant design challenge. Particularly challenging, is the design of structures configured for targeted delivery of its cargo to an individual without disruption to the surrounding living tissue of that individual. Previous efforts in this area have focused on polymer-based microcapsules sensitive to thermal gradients and pH. With advances in nanotechnology, incorporating nanomaterials of varied compositions and morphologies in polymer or gel-based matrices has led to the development of structures configured to remotely control the release process using stimuli such as electric and magnetic fields, as well as optical excitation. However, existing microcapsule cargo delivery systems are not suitable for controlled release applications in a living host since high power and temperatures are needed in order to release the encapsulated cargo, resulting in damage to the surrounding healthy tissue in the living host.

### SUMMARY

Self-assembled organic ligand functionalized microcapsules encapsulating one or more substrates, which release the substrates upon activation with a power source, are provided. Compositions that include these microcapsules, as well as methods of making the microcapsules and releasing the encapsulated substrates are also provided. The structures, compositions and methods find use in a variety of applications, such as drug and cell encapsulation technologies, for direct delivery, control, and activation of medicines and therapies to specific tissues in a living host e.g. targeted cancer therapy and pain management.

Aspects of the present disclosure include a self-assembled microcapsule composed of organic ligand-functionalized nanoparticles which contain one or more substrates encapsulated inside the microcapsule, wherein the microcapsule releases the substrate upon activation with a power source and the maximum temperature change at the microcapsule surface upon activation with the power source is 75° C. or less.

In some embodiments of the present disclosure, the organic ligand has the structure of formula (I):

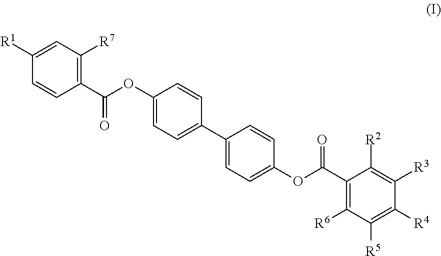

wherein
R¹ and R⁷ are each independently selected from, C₁-C₈ alkoxy, and C₁-C₈ alkoxy substituted with an amine or thiol group; and
R², R³, R⁴, R⁵ and R⁶ are each independently selected from H, halogen, hydroxyl, azido, alkyl, substituted alkyl, alkenyl, substituted alkenyl, alkynyl, substituted alkynyl, C₁-C₁₂ alkoxy, substituted alkoxy, amino, substituted amino, cycloalkyl, substituted cycloalkyl, heterocycloalkyl, substituted heterocycloalkyl, aryl, substituted aryl, heteroaryl, substituted heteroaryl, phosphate, substituted phosphate, phosphoryl, substituted phosphoryl, thiol and substituted thiol and combinations thereof. Aspects of the present disclosure also include a composition that includes a liquid, and a self-assembled microcapsule composed of organic ligand-functionalized nanoparticles containing one or more substrates encapsulated inside the microcapsule, in the liquid, wherein the organic ligand has a structure of formula (I). In some embodiments, the liquid is an organic solvent. In some embodiments, the liquid is a mesomorphic material, such as a liquid crystalline liquid. In some embodiments the liquid is a pharmaceutically acceptable liquid.

Other aspects of the present disclosure include a composition for producing a self-assembled microcapsule. The composition includes organic ligand-functionalized nanoparticles, and an anisotropic host phase, wherein the organic ligand has a structure of formula (I).

Further aspects of the present disclosure include a method of delivering one or more substrates to an individual. The method includes administering an effective amount of a self-assembled microcapsule composed of organic ligand-functionalized nanoparticles containing one or more substrates encapsulated inside the microcapsule; and applying activation from a power source to release the one or more substrates, wherein the maximum temperature change at the microcapsule surface upon activation with the power source is 75° C. or less. In some embodiments, the organic ligand has a structure of formula (I).

Other aspects of the present disclosure include a kit including a self-assembled microcapsule having a self-assembled microcapsule composed of organic ligand-functionalized nanoparticles and one or more substrates encapsulated inside the microcapsule and instructions for use. In some embodiments, the organic ligand has a structure of formula (I).

In some embodiments, the mean inter-particle separation of the nanoparticles in the self-assembled microcapsule is from 1 nm to 100 nm.

In some embodiments, the self-assembled microcapsule is composed of upconversion nanoparticles, plasmonic nanoparticles, or combinations thereof.

In some embodiments, the self-assembled microcapsule is composed of a nanoparticle material selected from a semiconductor material, a metal, a metal oxide, a metalloid, a metal coated material, an oxide, a magnetic material, a nanosome, a lipidsome and a polymer, or combinations thereof.

In some embodiments, the self-assembled microcapsule is composed of gold nanoparticles, silver nanoparticles, zinc oxide nanoparticles, gold coated nanoparticles, silver coated nanoparticles, zinc coated nanoparticles or combinations thereof.

In some embodiments, the self-assembled microcapsule is composed of iron oxide nanoparticles, cobalt nanoparticles, graphene coated iron oxide nanoparticles, graphene coated cobalt, silica coated iron oxide and silica coated cobalt or combinations thereof.

In some embodiments, the self-assembled microcapsule is composed of gold nanoparticles.

In some embodiments, the self-assembled microcapsule has a spherical surface. In some cases, the spherical surface has an average diameter of 100 nm to 100 μm. In particular embodiments, the spherical surface has an average diameter of 1 nm to 100 nm.

In some embodiments, the self-assembled microcapsule has nanoparticles functionalized with organic ligands selected from the following group:

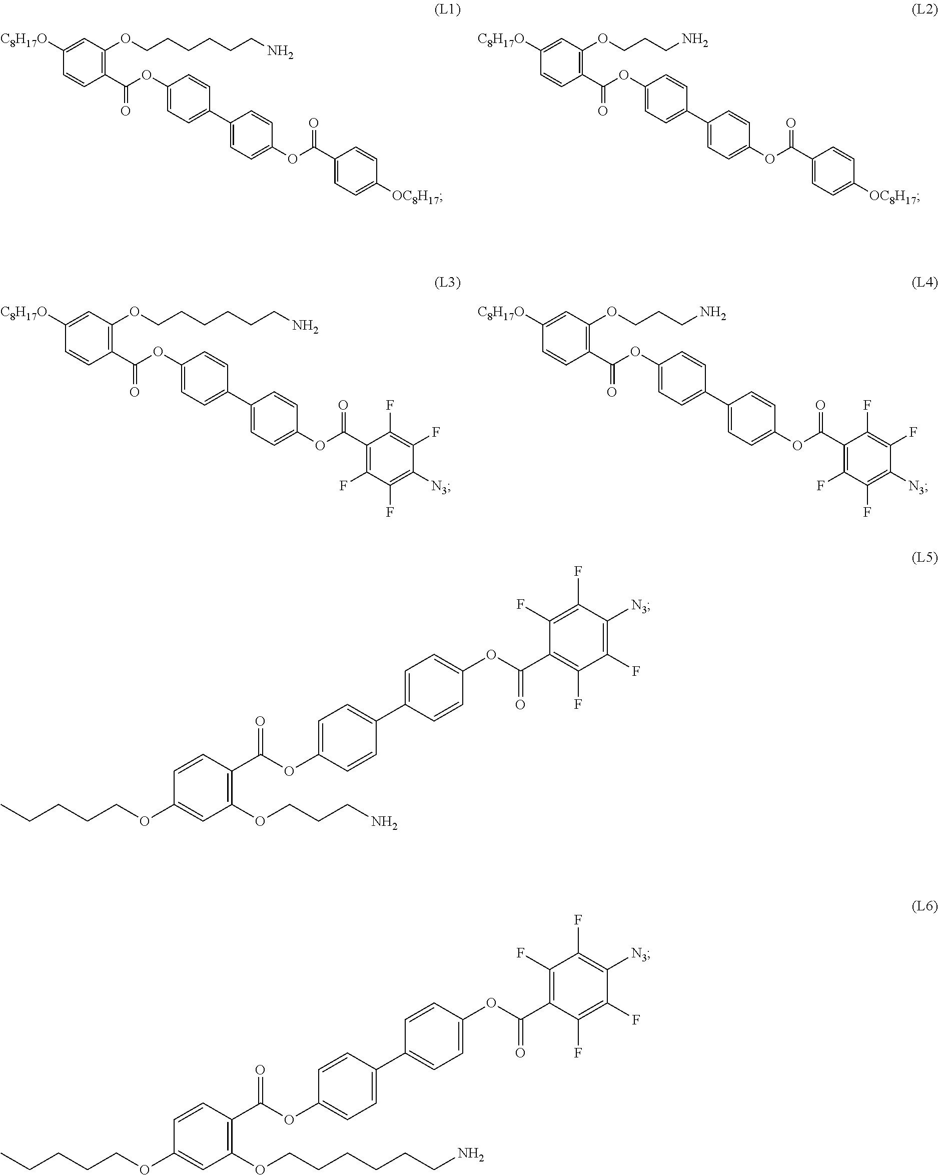

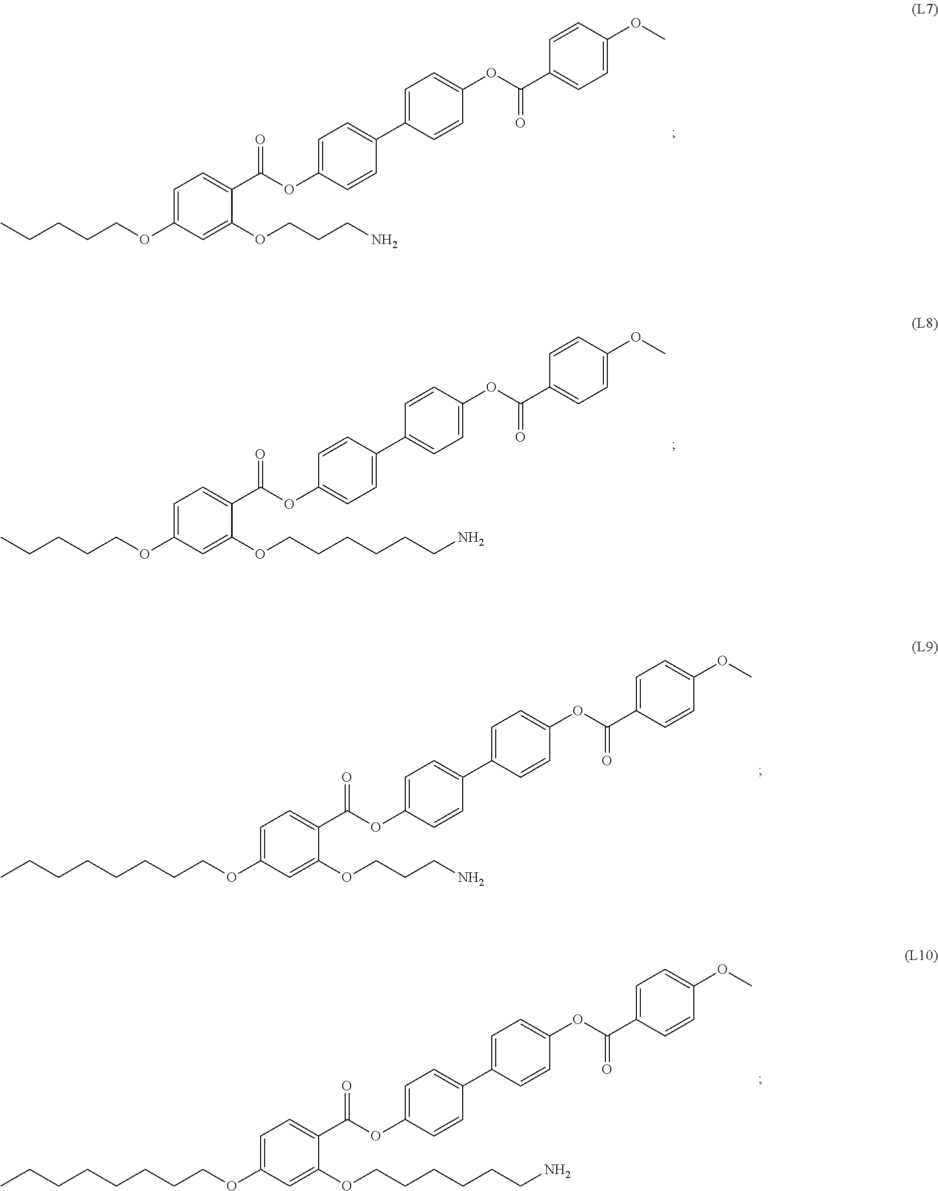

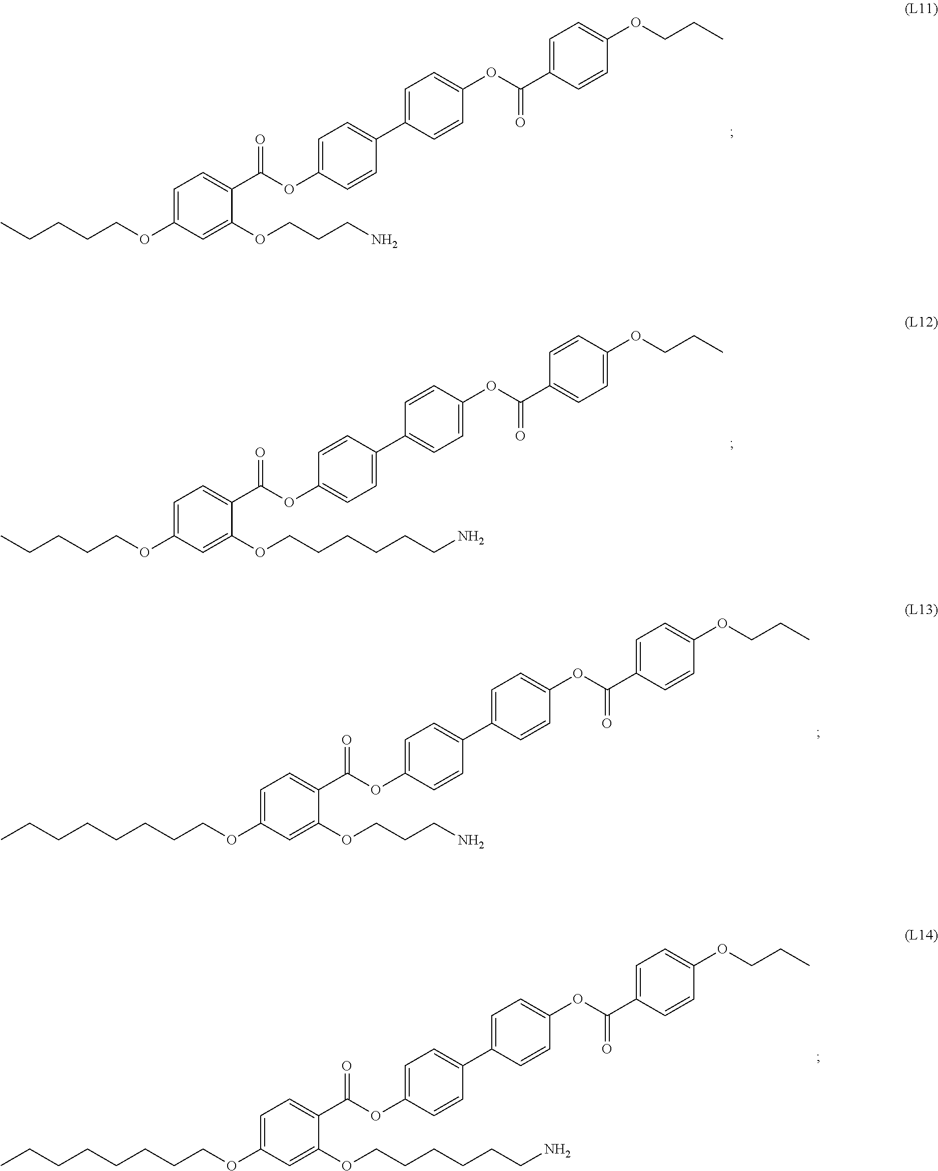

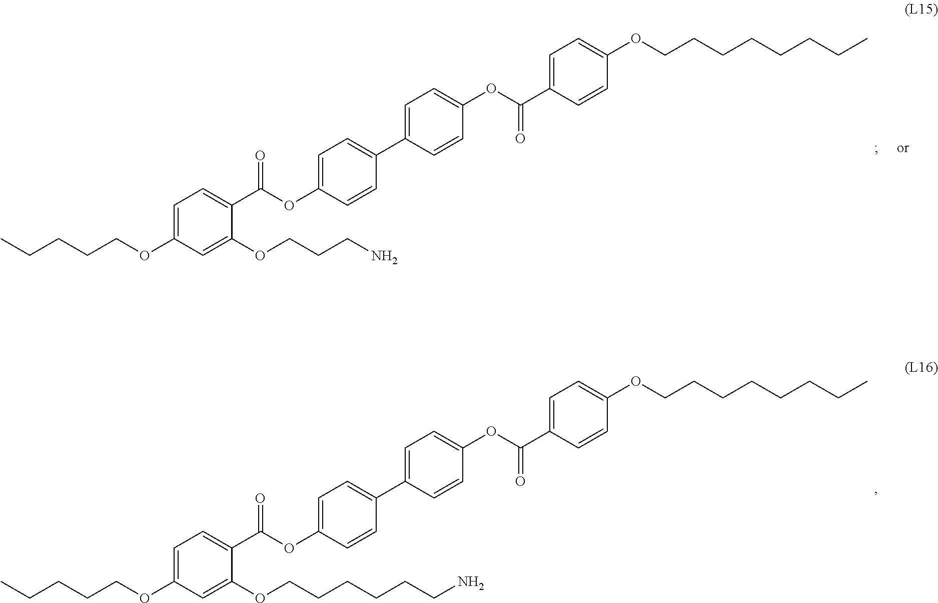

or combinations thereof.

In some embodiments, the self-assembled microcapsule has a thickness of from 1% to 50% of the volume of the microcapsule.

In some embodiments the substrate encapsulated within the self-assembled microcapsule is an active agent. In some cases, the substrate is live cells.

In some embodiments, the power source to release the substrate encapsulated within the self-assembled microcapsule is below the American National Standards Institute (ANSI) maximum permissible exposure limit. In some cases, the maximum permissible exposure power density of the power source is 100 mW/cm\hat{ }2 or less. In certain cases, the maximum permissible exposure time to the power source is 6 minutes or less.

In some embodiments, release of the substrate encapsulated within the self-assembled microcapsule is activated through localized surface plasmon resonance (LSPR) stimuli. In some cases, the release of the substrate is activated at an excitation wavelength of from 200 nm to 1 mm. In other cases, the release of the substrate is activated at an excitation wavelength of from 400 nm to 1 mm. In some embodiments release of the substrate is activated with a light that has a wavelength from 200 nm to 1 mm at a power density 100 mW/cm\hat{ }2 or less.

In some embodiments, full release of the substrate encapsulated within the self-assembled microcapsule is obtained in 6 minutes or less from the time of activation with a power source.

In some embodiments, the maximum temperature change at the surface of the self-assembled microcapsule upon activation with a power source is 75° C. or less. In some cases, the temperature change at the surface of the self-assembled microcapsule upon activation with a power source is 50° C. or less.

### BRIEF DESCRIPTION OF THE DRAWINGS

FIG. 1, panel (a), shows an intact multiple walled microcapsule with sectional cut-out to show the encapsulated dye within. FIG. 1, panel (b), shows illumination by green light, resonant with the localized surface plasmon resonance (LSPR) of the nanoparticles in the wall, disrupts the structure due to photothermal heating, releasing the contents within. FIG. 1, panel (c), shows Bright field microscopy image of AuNP in liquid crystal medium. FIG. 1, panel (d), shows a cross-polarized image of a AuNP in liquid crystal medium, extracted from suspension and placed between glass slides. FIG. 1, panel (e) is a close up of panel (f), showing an individual AuNP that forms the wall of the microcapsule.

FIG. 2, panels (a)-(e), depict the steps of the self-assembled microcapsule formation procedure.

FIG. 3, panels 1-5, show sequential images from polarized optical microscopy of the LC texture of a homeotropically aligned LC cell throughout the transition from isotropic to nematic phase. Additionally, the homeotropic alignment sample images shows point defects at the center and liquid crystal molecules aligned radially. FIG. 3, panel 6, shows a polarized optical microscopy image of the LC texture of a planar aligned LC cell. This image shows topological defects, such as saturn-ring defects and bipolar defects.

FIG. 4, panels (a)-(c), shows fluorescence images of Lumogen F Red encapsulated in a self-assembled microcapsule. FIG. 4, panel (d), shows dye intensity measured over five months. The inset shows dye intensity encapsulated in microcapsules compared to that of dye suspended in a liquid crystal alone.

FIG. 5, panels (1)-(2), shows a Bright field (BF) image and fluorescence (FL) image respectively of green fluorescence protein (GFP labelled E. coli bacterium captured within a microcapsule. FIG. 5, panel (3), shows 0.2 μm diameter fluorescent spheres in a gold microcapsule visualized in bright under a fluorescence microscope. The white scale bar represents 2 μm.

FIG. 6, panels (a)-(c), shows actuation leading to the release of the contents from a self-assembled microcapsule. FIG. 6, panel (a), shows fluorescence microscopy images of a self-assembled microcapsule loaded with a fluorescent dye on a temperature-controlled stage. The temperature was increased from 80 to 108° C., and the time after reaching 108° C. is given in the lower right corner. FIG. 6, panel (b), shows Bright-field and FIG. 6, panel (c), shows fluorescence time-lapse images during plasmon-actuated shell disintegration. The encapsulated dye is released during 5 s of illumination with 2 mW of incident power. Scale bars: 3 μm.

FIG. 7 shows the spectral dependence of photothermal bubble formation. FIG. 7, panel (a), shows that the release time τ decreases with increasing power for three different excitation wavelengths; the fastest release is achieved at 514 nm, which is the wavelength closest to the LSPR (520 nm). FIG. 7, panel (b), shows the equilibrium bubble radius R_{eq} increases with increasing power, and is largest at 514 nm. The inset shows a cross-polarized image of the bubble which exhibits isotropic phase inside and nematic phase outside. Scale bar: 3 μm. FIG. 7, panel (c), shows the bubble radius r(t) increases over the first 100 ms of excitation at each wavelength. FIG. 7, panel (d), shows simulated thermal maps over a range of excitation wavelengths showing that photothermal temperature changes remain strongly localized to the microcapsule surface. Scale bar: 1 μm. FIG. 7, panel (e), shows that the extinction spectrum of a microcapsule with resonance at 520 nm (curve) shows good agreement with the maximum temperature change at the shell surface (filled circles).

### TERMS

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except for the C₁ carbon) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂— alkyl, —SO₂-substituted alkyl, —SO₂-aryl, and —SO₂-heteroaryl.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring.

This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Thiol” refers to the group —SH.

The term “substituted thiol” refers to the group —SR where each R is independently selected from the group consisting of alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂— heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, —SO₂-heteroaryl, and fused heterocycle.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂— heteroaryl, and trihalomethyl.

Unless indicated otherwise, the nomenclature of substituents that are not explicitly defined herein are arrived at by naming the terminal portion of the functionality followed by the adjacent functionality toward the point of attachment. For example, the substituent “aminoalkoxy” refers to the group NH₂-(alkyl)-O—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

As used herein, the term “active agent” is meant to refer to compounds that are therapeutic agents. The term also refers to chemical and therapeutic agents including live materials.

The term “live cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

The term “mesomorphic material” refers to a material existing in a state of matter between liquid and crystal, such as a material that forms a mesomorphic state or mesophase. In some cases, the mesomorphic material is a liquid crystalline liquid or liquid crystal (LC). In some cases, the mesomorphic state or mesophase is a state or phase intermediate between that of the anisotropic crystal and that of the isotropic liquid. There are several mesomorphic states or forms, such as but not limited to, the semectic mesophase and the cholesterolic or nematic mesophase. In some cases the mesomorphic material is thermotropic.

### DETAILED DESCRIPTION

Self-assembled organic ligand functionalized microcapsules encapsulating one or more substrates, which release the substrates upon activation with a power source, are provided. Compositions that include these microcapsules, as well as methods of making the microcapsules and releasing the encapsulated substrates are also provided. The structures, compositions and methods find use in a variety of applications, such as drug and cell encapsulation technologies, for direct delivery, control, and activation of medicines and therapies to specific tissues in an individual.

### Self-Assembled Microcapsules

Aspects of the present disclosure include self-assembled microcapsules composed of organic ligand functionalized nanoparticles. By “nanoparticles” is meant particles that have a size range in the nanometer (nm) scale. For example, a nanoparticle may have a size (e.g., largest dimension) of 1000 nm or less, such as a size ranging from 0.1 nm to 1000 nm. Self-assembled microcapsules of the present disclosure include structures having a shape that extends in three dimensions, such as length, width and height. Three-dimensional structures are distinct from one-dimensional structures (e.g., linear structures) and two-dimensional structures (e.g., planar structures).

The self-assembled microcapsules of the present disclosure include structures having a shell configuration. The term “shell” or “shell configuration” as used herein describes structures where a surface at least partially, and sometimes completely, encloses a space or material. A shell or shell configuration may also be referred to as a “capsule” or “microcapsule”. A shell may partially or completely enclose the space or material. For instance, a shell may partially enclose the space or material, such as enclose 50% or more of the space or material, or 60% or more, or 70% or more, or 80% or more, or 90% or more, or 95% or more, or 97% or more, or 99% or more of the space or material. Partial enclosure of a space or material includes embodiments where the surface is substantially contiguous and has one or more voids (e.g., holes) in the surface, and also includes embodiments where the surface is substantially continuous but the surface does not extend to completely enclose the space or material. In other embodiments, the shell completely encloses the space or material, such that the surface is substantially continuous without significant discontinuities (e.g., voids or holes) in the surface.

Surfaces with a shell configuration may have various shapes and sizes. For instance, shell configurations include, but are not limited to, regular shapes such as spherical shells, ellipsoid shells, cylinder shells, cone shells, cube shells, cuboid shells, pyramidal shells, torus shells, and the like. In other embodiments, the shell may have an irregular shape. In certain embodiments, structures of the present disclosure have a shell configuration, where the shell configuration is a spherical surface (i.e., a spherical shell). By “microcapsule” or “microcapsule configuration” is meant the structure has a size range in the micrometer (μm) scale. For example, a microstructure may have a size (e.g., largest dimension) of 100 μm or less, such as a size ranging from 100 nm to 100 μm (0.1 μm to 100 μm), 1 nm to 100 nm (0.001 μm to 0.1 μm).

In certain embodiments, the structures are microcapsules as described above, where the microcapsule have a size of 1000 μm or less, such as 950 μm or less, or 900 μm or less, or 850 μm or less, or 800 μm or less, or 750 μm or less, or 700 μm or less, or 650 μm or less, or 600 μm or less, or 550 μm or less, or 500 μm or less, or 450 μm or less, or 400 μm or less, or 350 μm or less, or 300 μm or less, or 250 μm or less, or 200 μm or less, or 150 μm or less, or 100 μm or less, or 90 μm or less, or 80 μm or less, or 70 μm or less, or 60 μm or less, or 50 μm or less, or 40 μm or less, or 30 μm or less, or 20 μm or less, or 10 μm or less, or 9 μm or less, or 8 μm or less, or 7 μm or less, or 6 μm or less, or 5 μm or less, or 4 μm or less, or 3 μm or less, or 2 μm or less, or 1 μm or less, or 0.75 μm or less, or 0.5 μm or less, or 0.25 μm or less, or 0.1 μm or less, or 0.075 μm or less, or 0.05 μm or less, or 0.025 μm or less, or 0.01 μm or less. In some instances, the microcapsules have a size ranging from 0.01 μm to 1000 μm, 0.025 μm to 1000 μm, 0.05 μm to 1000 μm, 0.075 μm to 1000 μm, 0.1 μm to 1000 μm, such as from 0.25 μm to 1000 μm, or 0.5 μm to 1000 μm, or 0.5 μm to 900 μm, or 0.5 μm to 800 μm, or 0.5 μm to 700, or 0.5 μm to 600 μm, or 0.5 μm to 500 μm, or 0.5 μm to 400 μm, or 0.5 μm to 300 μm, or 0.5 μm to 250 μm, or 0.5 μm to 200 μm, or 0.5 μm to 150 μm, or 0.5 μm to 100 μm, or 0.5 μm to 90 μm, or 0.5 μm to 80 μm, or 0.5 μm to 70 μm, or 0.5 μm to 60 μm, or 0.5 μm to 50 μm, or 0.5 μm to 40 μm, or 0.5 μm to 30 μm, or 0.5 μm to 20 μm, or 0.5 μm to 10 μm, or 0.5 μm to 9 μm, or 0.5 μm to 8 μm, or 0.5 μm to 7 μm, or 0.5 μm to 6 μm, or 0.5 μm to 5 μm, or 0.5 μm to 4 μm, or 0.5 μm to 3 μm, or 0.5 μm to 2 μm, or 0.5 μm to 1 μm. In some instances, the microcapsules have a size ranging from 0.001 μm to 0.01 μm, 0.002 μm to 0.01 μm, 0.003 μm to 0.01 μm, 0.004 μm to 0.01 μm, 0.005 to 0.01 μm, or 0.006 μm to 0.01 μm, or 0.007 μm to 0.01, or 0.008 μm to 0.01 μm, or 0.009 μm to 0.01 μm, or 0.005 μm to 0.003 μm, or 0.005 μm to 0.002 μm, or 0.005 μm to 0.001 μm. The size of the microcapsules may be measured as the largest dimension of the microcapsule (e.g., length, width, or height), or for spherical microstructures (e.g., spherical surfaces), may be measured as the average diameter of the microstructures. By “average” is meant the arithmetic mean. In some embodiments, the microcapsules have an average diameter of from 100 nm to 100 μm. In some embodiments, the microcapsules have an average diameter of from 1 nm to 100 nm. In certain instances, the microcapsules have an average size of 5 μm. In certain instances, the microcapsules have an average size of 1 μm. In certain instances, the microstructures have an average size of 0.1 μm. In certain instances, the microstructures have an average size of 0.05 μm. Mixtures of different sizes and/or shapes of self-assembled microcapsules may be used as desired. In other embodiments, the self-assembled microcapsules have substantially the same size and shape. In some cases, the microcapsule has a thickness of from 1% to 50% of the volume of the microcapsule. By “thickness”, it is meant the thickness of the shell.

The self-assembled microcapsules of the present disclosure are composed of organic ligand functionalized nanoparticles. In certain embodiments, the nanoparticles are stably associated with each other to form the shell. By “stably associated” is meant that a moiety is bound to or otherwise associated with another moiety or structure under standard conditions. In certain instances, the nanoparticles may be stably associated with each other such that the shell substantially maintains its shape after formation of the shell. In some embodiments, the nanoparticles are stably associated with each other through non-covalent interactions, such as, but not limited to, ionic bonds, hydrophobic interactions, hydrogen bonds, van der Waals forces (e.g., London dispersion forces), dipole-dipole interactions, and the like. In some embodiments, the nanoparticles are stably associated with each other through covalent bonds. For example, a nanoparticle may be covalently bound or cross-linked to one or more nanoparticles in the shell. In certain cases, the nanoparticles are stably associated with each other through a combination of non-covalent and covalent interactions.

As described above, the self-assembled microcapsules of the present disclosure may be composed of nanoparticles. The nanoparticles may have a size of 1000 nm or less, such as 900 nm or less, or 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 90 nm or less, or 80 nm or less, or 70 nm or less, or 60 nm or less, or 50 nm or less, or 40 nm or less, or 30 nm or less, or 20 nm or less, or 10 nm or less, or 9 nm or less, or 8 nm or less, or 7 nm or less, or 6 nm or less, or 5 nm or less, or 4 nm or less, or 3 nm or less, or 2 nm or less, or 1 nm or less. In some instances, the nanoparticles have a size ranging from 0.1 nm to 1000 nm, such as from 0.5 nm to 1000 nm, or 1 nm to 1000 nm, or 1 nm to 900 nm, or 1 nm to 800 nm, or 1 nm to 700 nm, or 1 nm to 600 nm, or 1 nm to 500 nm, or 1 nm to 400 nm, or 1 nm to 300 nm, or 1 nm to 250 nm, or 1 nm to 200 nm, or 1 nm to 150 nm, or 1 nm to 100 nm, or 1 nm to 90 nm, or 1 nm to 80 nm, or 1 nm to 70 nm, or 1 nm to 60 nm, or 1 nm to 50 nm, or 1 nm to 40 nm, or 1 nm to 30 nm, or 1 nm to 20 nm, or 1 nm to 10 nm, or 1 nm to 9 nm, or 1 nm to 8 nm, or 1 nm to 7 nm, or 1 nm to 6 nm, or 1 nm to 5 nm. The size of the nanoparticles may be measured as the largest dimension of the nanoparticle (e.g., length, width, etc.), or for spherical nanoparticles, may be measured as the average diameter of the nanoparticles. In certain instances, the nanoparticles have an average size of 5 nm. In certain instances, the nanoparticles have an average size of 6 nm. Mixtures of different sizes and/or shapes of nanoparticles may be included in the three-dimensional structures as desired. In other embodiments, the nanoparticles have substantially the same size and shape.

Nanoparticles may have various shapes, such as, but not limited to, spherical, ellipsoid, cylinder, cone, cube, cuboid, pyramidal, needle, and the like. The nanoparticles may be made of any convenient material, such as, but not limited to, upconversion nanoparticles, plasmonic nanoparticles, or combinations thereof. The nanoparticles may be made of a semiconductor material, a metal, a metal oxide, a metal coated material, a metalloid, an oxide, a magnetic material, a nanosome, a lipidsome, a polymer, combinations thereof, and the like. For example, nanoparticles may be composed of materials, such as, but not limited to, titanium dioxide, silicon, gold, gold-plated silica, polymers, silver, zinc oxide, iron oxide, cobalt and the like. In some cases, the nanoparticles may be composed of coated nanoparticles, such as polymer-coated, gold coated, silver coated, zinc coated, graphene coated, graphene coated cobalt, silica coated iron oxide, silica coated cobalt nanoparticles, and the like. In some embodiments the nanoparticles are gold nanoparticles.

In certain embodiments, the nanoparticles that form the self-assembled microcapsule are arranged as a mixture of nanoparticles to form the three-dimensional structure. For instance, the microcapsule may be composed of a mixture (e.g., a substantially homogeneous mixture) of nanoparticles. In some embodiments, the nanoparticles are arranged in one or more layers to form the microcapsule. The composition of each layer of the microcapsule may be the same or may be different. For example, each layer of the microcapsule may be composed of the same type of nanoparticle or mixture of nanoparticles. Nanoparticles that are of the same type may include nanoparticles that are substantially the same with respect to their physical and chemical characteristics, such as, but not limited to, size, shape, composition, organic ligand attached to the surface of the nanoparticle, and the like. In other cases, a layer of the microcapsule may have a different composition (e.g., a different nanoparticle or mixture of nanoparticles) than an adjacent layer. For instance, nanoparticles may differ with respect to one or more physical and/or chemical characteristics, such as, but not limited to, size, shape, composition, organic ligand attached to the surface of the nanoparticle, and the like.

In certain embodiments, the self-assembled microcapsule is composed of nanoparticles where the nanoparticles are a mixture of different types of nanoparticles. For instance, the mixture of nanoparticles may be a heterogeneous mixture of nanoparticles that is composed of different types of nanoparticles. The different types of nanoparticles may include nanoparticles that vary in one or more physical and/or chemical characteristics, such as, but not limited to, size, shape, composition, organic ligand attached to the surface of the nanoparticle, combinations thereof, and the like.

In certain embodiments, the nanoparticle is composed of a material or mixture of materials, such that the composition of the nanoparticle is substantially homogeneous. In some cases, the nanoparticle is composed of two or more materials. Nanoparticles composed of two or more materials include nanoparticles composed of a mixture of the two or more materials, such that the nanoparticles have a substantially homogeneous composition, and nanoparticles where the nanoparticles are composed of regions of a material interspersed with or adjacent to regions of one or more different materials. For instance, a nanoparticle may be composed of a core of a first material (or mixture of materials) substantially surrounded by a shell of a different material (or different mixture of materials). The shell of the different material may be disposed as one or more layers of material on a surface of the core of the first material.

The nanoparticles of the present disclosure are organic ligand-functionalized nanoparticles. An organic ligand-functionalized nanoparticle is a nanoparticle that includes an organic ligand attached to the surface of the nanoparticle. The ligand may be attached to the surface of the nanoparticle through non-covalent interactions, such as, but not limited to, ionic bonds, hydrophobic interactions, hydrogen bonds, van der Waals forces (e.g., London dispersion forces), dipole-dipole interactions, and the like, or through covalent bonds. In certain embodiments, the ligand is attached to the surface of the nanoparticle through a covalent bond.

Organic ligands suitable for functionalization of the nanoparticles may vary depending on the desired properties of the functionalized nanoparticle. For example, the organic ligand on the ligand-functionalized nanoparticle may be selected such that the spacing between adjacent ligand-functionalized nanoparticles is a desired spacing. Stated another way, in some instances, the spacing between adjacent organic ligand-functionalized nanoparticles may depend on one or more properties of the organic ligand, such as, but not limited to, the size, structure, and/or orientation of the ligand. In some cases, the spacing between adjacent nanoparticles is 1 nm or more, such as 2 nm or more, 3 nm or more, 4 nm or more, 5 nm or more, 6 nm or more, or 7 nm or more, or 8 nm or more, 9 nm or more, 10 nm or more, 11 nm or more, 12 nm or more, 13 nm or more, 14 nm or more, 15 nm or more, 16 nm or more, 17 nm or more, 18 nm or more, 19 nm or more, or 20 nm or more. In some cases, the spacing between adjacent nanoparticles is 20 nm or more, such as 25 nm or more, 30 nm or more, 35 nm or more, 40 nm or more, 45 nm or more, 50 nm or more, 55 nm or more, 60 nm or more, 65 nm or more, 70 nm or more, 75 nm or more, 80 nm or more, 85 nm or more, 90 nm or more, 95 nm or more, 100 nm or more. In some cases, the spacing between adjacent nanoparticles is 10 nm or more. In some cases, the spacing between adjacent nanoparticles is 5 nm to 20 nm, such as 7 nm to 15 nm, or 10 nm to 15 nm. In some instances, the spacing between adjacent nanoparticles is 10 nm to 15 nm, such as 10 nm to 13 nm, or 10 nm to 12 nm. In some cases, the mean inter-particle separation of the nanoparticles is from 1 nm to 100 nm. In certain embodiments, the spacing between adjacent nanoparticles is selected so as to minimize shifts in the emission spectrum of the nanoparticles. In certain embodiments, the spacing between adjacent nanoparticles is selected so as to minimize energy losses due to fluorescence resonance energy transfer (FRET).

In some embodiments, the organic ligand disclosed herein has mesomorphic state properties, such as liquid crystalline properties. For instance, an organic ligand may include a rigid moiety and one or more flexible moieties. The rigid and flexible moieties of the organic ligands may facilitate alignment of the organic ligands in a common direction. For example, as described herein, organic ligand-functionalized nanoparticles may be dispersed in a mesomorphic material, such as a liquid crystalline liquid, and thus the flexible moiety may facilitate alignment of the organic ligand with the surrounding mesomorphic material. For instance, organic ligands attached to a surface of a nanoparticle may align with the director of a surrounding mesomorphic material (e.g., a nematic phase or mesomorphic state of the mesomorphic material).

In certain embodiments, the organic ligand has a phase transition temperature (also referred to as a melting temperature or clearing point) ranging from 50° C. to 150° C., such as 75° C. to 125° C., or 80° C. to 120° C., or 85° C. to 115° C., or 90° C. to 110° C. In certain embodiments, the organic ligand has a phase transition temperature (e.g., melting temperature or clearing point) of about 100° C., such as 90° C., 91° C., 92° C., 93° C., 94° C., 95° C., 96° C., 97° C., 98° C., 99° C. or 100° C. For example, the phase transition temperature may be a temperature at which the organic ligand transitions from a first phase to a second phase (or vice versa). In some embodiments, the organic ligand may transition from a phase having positional order (e.g., an ordered spatial arrangement of the ligands, such as in an ordered lattice) or directional order (e.g., alignment of the ligands along a common directional axis) to a phase having substantially no positional or directional order. In some embodiments, the organic ligand may transition from a phase having substantially no positional or directional order to a phase having positional or directional order. In some cases, the organic ligand has positional and/or directional order below the phase transition temperature, and substantially no positional or directional order above the phase transition temperature. Similarly, organic ligands that are stably associated with or attached to a surface of organic ligand-functionalized nanoparticles may have a phase transition from a phase having substantially no positional or directional order to a phase having positional or directional order (or vice versa). As described above, organic ligands that are stably associated with or attached to a surface of organic ligand-functionalized nanoparticles may have a phase transition temperature (also referred to as a melting temperature or clearing point) ranging from 50° C. to 150° C., such as 75° C. to 125° C., or 80° C. to 120° C., or 85° C. to 115° C., or 90° C. to 110° C. In certain embodiments, organic ligands that are stably associated with or attached to a surface of organic ligand-functionalized nanoparticles may have a phase transition temperature (e.g., melting temperature or clearing point) of about 100° C., such as 90° C., 91° C., 92° C., 93° C., 94° C., 95° C., 96° C., 97° C., 98° C., 99° C. or 100° C.

In certain embodiments, the organic ligands include aromatic rings. In certain cases, the organic ligands have a structure according to formula (I). In certain cases, the organic ligands have a structure according to any of (L1) to (L16) as defined herein. In certain cases, the organic ligands have the structure of (L1). In certain cases, the organic ligands have the structure of (L2). In certain cases, the organic ligands have the structure of (L3). In certain cases, the organic ligands have the structure of (L4). In certain cases, the organic ligands have the structure of (L5). In certain cases, the organic ligands have the structure of (L6). In certain cases, the organic ligands have the structure of (L7). In certain cases, the organic ligands have the structure of (L8). In certain cases, the organic ligands have the structure of (L9). In certain cases, the organic ligands have the structure of (L10). In certain cases, the organic ligands have the structure of (L11). In certain cases, the organic ligands have the structure of (L12). In certain cases, the organic ligands have the structure of (L13). In certain cases, the organic ligands have the structure of (L14). In certain cases, the organic ligands have the structure of (L15). In certain cases, the organic ligands have the structure of (L16). In some embodiments, organic ligands suitable for functionalization of the nanoparticles are substituted alkyl groups. In some cases, the organic ligands include, but are not limited to, octadecylamine (ODA), octadecylphosphonic acid, oleic acid, combinations thereof, and the like.

In certain embodiments, the organic ligand includes a cross-linkable functional group. The cross-linkable functional group may be a group that, when activated, can form an attachment to another moiety. In some cases, the attachment may attach an organic ligand to another organic ligand (e.g., an organic ligand of an adjacent organic ligand-functionalized nanoparticle), may attach an organic ligand to a nanoparticle, may attach an organic ligand comprising an aromatic group to an organic ligand comprising a substituted alkyl group. In certain embodiments, the cross-linkable functional group forms a covalent bond attachment the other moiety. In certain embodiments, the cross-linkable functional group is a light activated cross-linkable functional group. A light activated cross-linkable functional group is a cross-linkable functional group that may form an attachment to another moiety when light is applied to the light activated cross-linkable functional group. For example, exposure of the light activated cross-linkable functional group to light may activate the functional group, thus forming a reactive moiety capable of forming a crosslink to another moiety as described above. In some instances, the applied light is ultraviolet (UV) light. In some instances, the applied light is visible light. In some instances, the applied light is infrared light. For example, the applied light may be UV light having a wavelength ranging from 100 nm to 400 nm, such as 150 nm to 400 nm, or 200 nm to 400 nm, or 300 nm to 400 nm. In some instances, the applied UV light may be approximately 350 nm, such as 360 nm or 364 nm. Other types of cross-linkable functional groups may also be used, such as chemically activated cross-linkable functional groups, and the like.

Any convenient cross-linkable functional group may be used. In certain embodiments, the cross-linkable functional group is a functional group that, when activated, forms a reactive moiety. The reactive moiety may then react with another moiety (e.g., organic ligand, nanoparticle, etc.) to form an attachment (e.g., covalent bond) between the cross-linkable functional group and the other moiety. In some cases, the reactive moiety is a moiety capable of forming a covalent bond to carbon. For example, the reactive moiety may be a nitrene, such as a reactive nitrene derived from an azide functional group (e.g., an azide cross-linkable functional group). A nitrene may form a covalent bond to carbon to produce an amine or amide. In some instances, the cross-linkable functional group includes an azide, such as, but not limited to, a tetrafluoro-arylazide group.

In some embodiments, organic ligand has a structure according to formula (I):

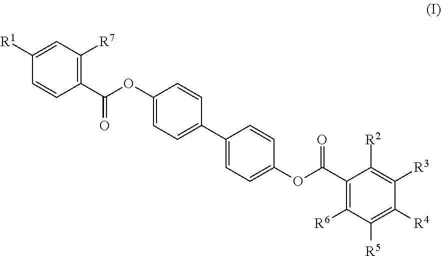

wherein

R¹ and R⁷ are each independently selected from, C₁-C₈ alkoxy, and C₁-C₈ alkoxy substituted with an amine or thiol group; and

R², R³, R⁴, R⁵ and R⁶ are each independently selected from H, halogen, hydroxyl, azido, alkyl, substituted alkyl, alkenyl, substituted alkenyl, alkynyl, substituted alkynyl, C₁-C₁₂ alkoxy, substituted alkoxy, amino, substituted amino, cycloalkyl, substituted cycloalkyl, heterocycloalkyl, substituted heterocycloalkyl, aryl, substituted aryl, heteroaryl, substituted heteroaryl, phosphate, substituted phosphate, phosphoryl, substituted phosphoryl, thiol, substituted thiol or combinations thereof.

In some instances, R⁴ to R⁸ are each independently selected from H, halo, azido, alkyl, substituted alkyl, alkoxy, and substituted alkoxy.

In some instances, R¹ is alkoxy, such as a C₁₋₈ alkoxy, C₁₋₆ alkoxy, or C₁₋₃ alkoxy. In some instances, R¹ is C₅ alkoxy, such as pentyloxy. In some instances, R¹ is C₈ alkoxy, such as octyloxy. In some instances, R¹ is C₃ alkoxy, such as propyloxy. In some embodiments, R¹ is C₁₋₈ alkoxy substituted with an amine group, such as C₁₋₆ alkoxy, or C₁₋₃ alkoxy substituted with an amine group. In some instances, R¹ is C₈ alkoxy substituted with an amine group. In some instances, R¹ C₆ alkoxy substituted with an amine group. In some instances, R¹ is C₆ alkoxy substituted with an amine group. In some instances the amine group is a primary amine. Said another way, in some embodiments R¹ is aminoalkoxy, such as aminopropoxy (e.g., 3-aminopropoxy) or aminohexyloxy (e.g., 6-aminohexyloxy). In some embodiments, R¹ is C₁₋₈ alkoxy substituted with a thiol group, such as C₁₋₆ alkoxy, or C₁₋₃ alkoxy substituted with a thiol group. In some instances, R¹ is C₈ alkoxy substituted with a thiol. In some instances, R¹ is C₆ alkoxy substituted with a thiol group. In some instances, R¹ is C₄ alkoxy substituted with a thiol group. In some instances, R¹ is H.

In some instances, R⁷ is alkoxy, such as a C₁₋₈ alkoxy, C₁₋₆ alkoxy, or C₁₋₃ alkoxy. In some instances, R⁷ is C₅ alkoxy, such as pentyloxy. In some instances, R⁷ is C₈ alkoxy, such as octyloxy. In some instances, R⁷ is C₃ alkoxy, such as propyloxy. In some embodiments, R⁷ is C₁₋₈ alkoxy substituted with an amine group, such as C₁₋₆ alkoxy, or C₁₋₃ alkoxy substituted with an amine group. In some instances, R⁷ is C₈ alkoxy substituted with an amine group. In some instances, R⁷ is C₆ alkoxy substituted with an amine group. In some instances, R⁷ is C₆ alkoxy substituted with an amine group. In some instances the amine group is a primary amine. Said another way, in some embodiments R⁷ is aminoalkoxy, such as aminopropoxy (e.g., 3-aminopropoxy) or aminohexyloxy (e.g., 6-aminohexyloxy). In some embodiments, R⁷ is C₁₋₈ alkoxy substituted with a thiol group, such as C₁₋₆ alkoxy, or C₁₋₃ alkoxy substituted with a thiol group. In some instances, R⁷ is C₈ alkoxy substituted with a thiol. In some instances, R⁷ is C₆ alkoxy substituted with a thiol group. In some instances, R⁷ is C₄ alkoxy substituted with a thiol group. In some instances, R⁷ is H.

In some embodiments, the organic ligand is attached to a nanoparticle through either the R¹ or R⁷ substituent. For instance, in embodiments where R¹ is an aminoalkoxy group, the organic ligand may be attached to the nanoparticle through the amino group of the aminoalkoxy.

In some instances, R² is H or halo. In some instances, R² is H. R² is halo, such as fluoro.

In some instances, R⁶ is H or halo. In some instances, R⁶ is H. R⁶ is halo, such as fluoro.

In some instances, R³ is H or halo. In some instances, R³ is H. R³ is halo, such as fluoro.

In some instances, R⁵ is H or halo. In some instances, R⁵ is H. R⁵ is halo, such as fluoro.

In some instances, R⁴ is alkoxy or azido. In some instances, R⁴ is azido. In some instances, R⁴ is alkoxy, such as a C₁₋₈ alkoxy, C₁₋₆ alkoxy, or C₁₋₃ alkoxy. In some instances, R⁴ is methoxy. In some instances, R⁴ is C₃ alkoxy, such as propoxy. In some instances, R⁴ is C₈ alkoxy, such as octyloxy. In some instances, R⁴ is substituted alkoxy, such as a substituted C₁₋₁₄ alkoxy, substituted C₁₋₁₂ alkoxy, substituted C₁₋₁₀ alkoxy, substituted C₁₋₈ alkoxy, or substituted C₁₋₆ alkoxy. In some instances, R⁴ is substituted C₆ alkoxy, such as substituted hexyloxy. In some instances, R⁴ is substituted C₁₀ alkoxy, such as substituted decyloxy. In some instances, the substituent on the substituted alkoxy is phosphate or substituted phosphate.

In some instances, R², R³, R⁵ and R⁶ are each H. In some instances, when R², R³, R⁵ and R⁶ are each H, R⁴ is alkoxy or substituted alkoxy.

In some instances, R², R³, R⁵ and R⁶ are each halo, such as fluoro. In some instances, when R², R³, R⁵ and R⁶ are each halo (e.g., fluoro), R⁴ is azido.

In certain embodiments, the organic ligand has a structure according to any of (L1) to (L16).

In some embodiments, the self-assembled microcapsule has nanoparticles functionalized with organic ligands selected from the following group:

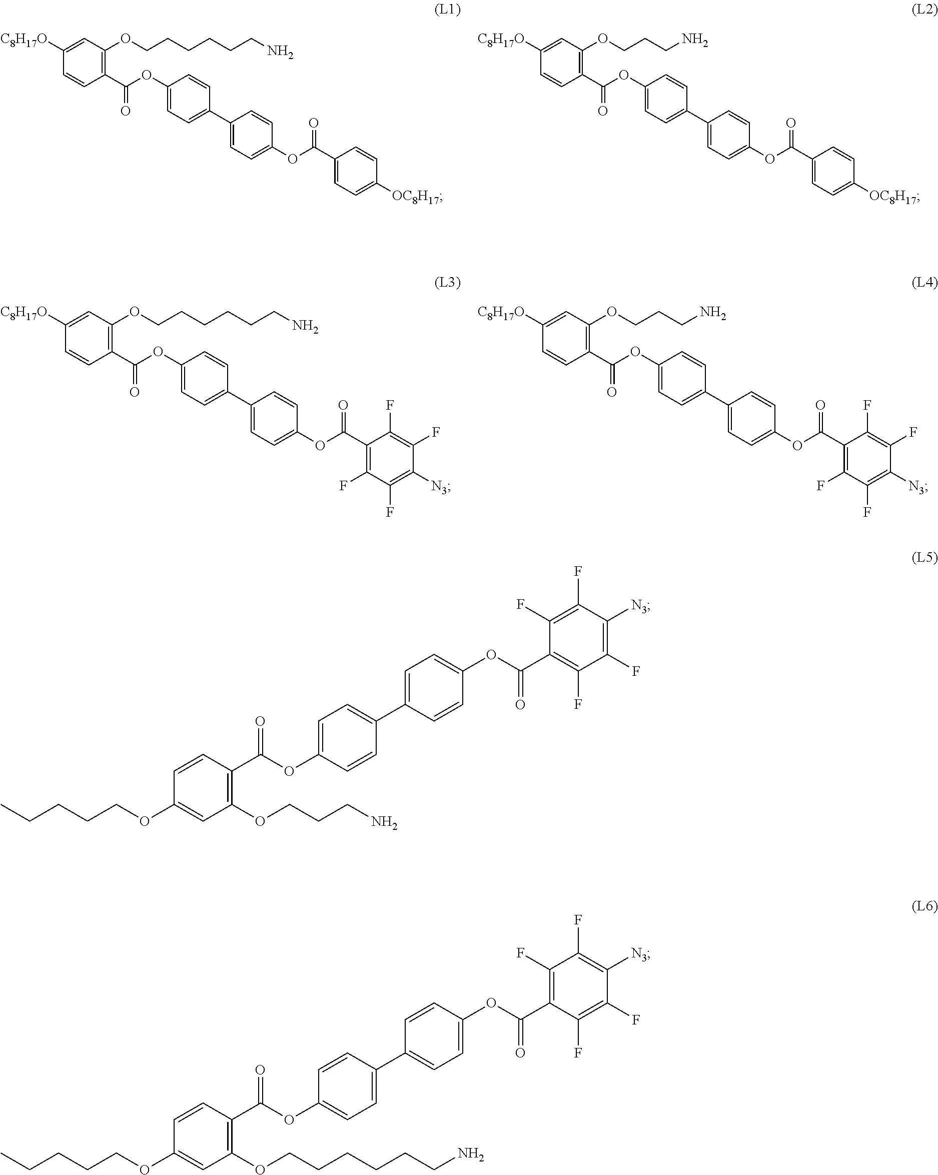

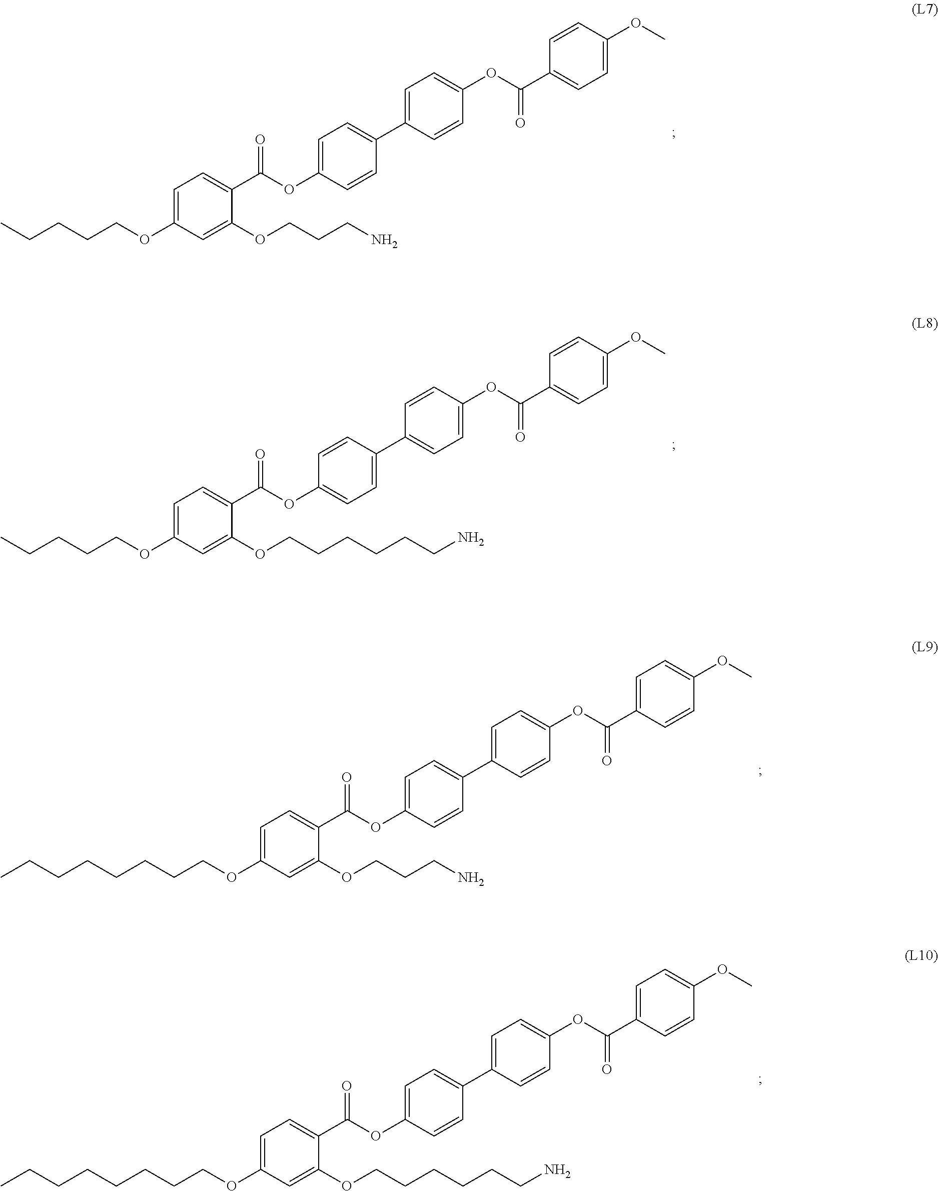

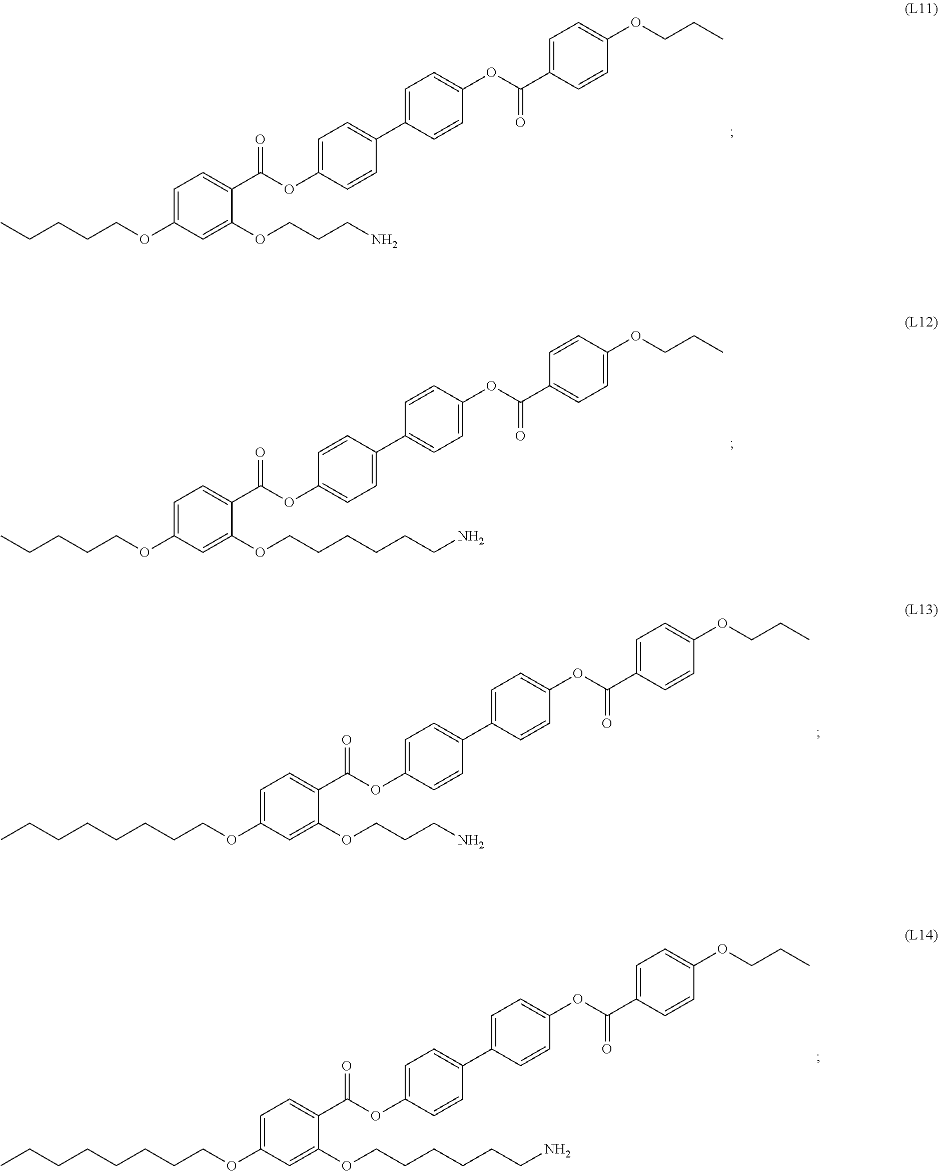

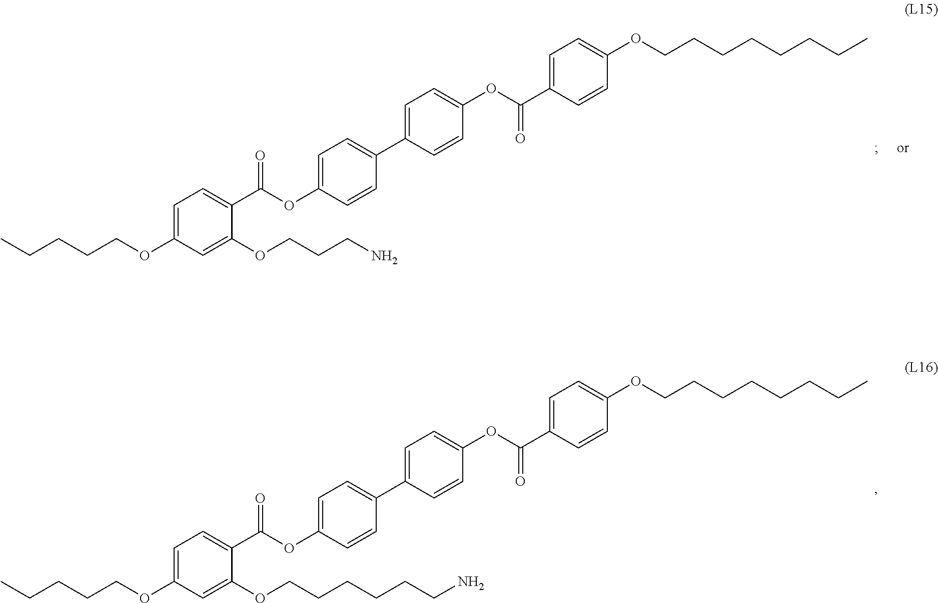

or combinations thereof.

As described above, the self-assembled microcapsules may be composed of nanoparticles having substantially the same physical and chemical characteristics, or in other embodiments, may be composed of nanoparticles having different physical and/or chemical characteristics. For example, physical and/or chemical characteristics of the nanoparticles that may be the same or may vary as described above may include, but are not limited to, size, shape, composition, ligand attached to the surface of the nanoparticle, organic ligand attached to the surface of the nanoparticle, cross-linkable functional group, combinations thereof, and the like. For instance, a nanoparticle may include a plurality of organic ligands attached to the surface of the nanoparticle, where the ligands are substantially the same. In other instances, the nanoparticle may include a plurality of ligands attached to the surface of the nanoparticle, where the ligands are different (e.g., ligands having different chemical structures and/or functional groups, such as cross-linkable functional groups as described herein). For example, combinations of various ligands may be attached to the surface of the same nanoparticle.

### Compositions

As described above, self-assembled microcapsules of the present disclosure may have a shell configuration that partially or completely encloses a space or material. In certain embodiments, the shell encloses a material, such as an active agent or live cells. In some instances, the active agent is a drug. Encapsulation of the active agent or live cells inside the microcapsule may facilitate one or more of: delivery of the active agent or live cells to a desired site; formulation of the active agent or live cells into a desired formulation; increased stability of the active agent or live cells; controlled release of the active agent or live cells; delayed release of the active agent or live cells; and the like.

Aspects of the present disclosure include compositions that include the self-assembled microcapsules as disclosed herein. The composition may include the self-assembled microcapsule and a liquid. In some instances, the composition includes the self-assembled microcapsule with a substrate encapsulated within dispersed in the liquid. In some instances, the liquid is a liquid crystalline fluid (e.g., a liquid crystalline liquid), such as a liquid crystalline liquid as described in more detail below. In some instances, the liquid is a solvent. In some embodiments the liquid is a pharmaceutically acceptable liquid. Any convenient solvent may be used, depending on the desired composition of the self-assembled microcapsule. Examples of solvents include, but are not limited to, organic solvents, such as toluene, dimethylbenzene, methylisopropylbenzene, methanol, ethyl acetate, chloroform, mixtures thereof, and the like. In some instances, the solvent is toluene.

Aspects of the present disclosure also include compositions for producing a self-assembled microcapsule of stably associated organic ligand-functionalized nanoparticles described herein. In certain embodiments, the composition includes organic ligand-functionalized nanoparticles and an anisotropic host phase (e.g., a liquid crystalline liquid). The nanoparticles in the composition for producing the self-assembled microcapsules may be any of the nanoparticles as described herein. For instance, the nanoparticles may be organic ligand-functionalized gold nanoparticles, as described herein.

In certain cases, the composition includes a liquid crystalline fluid (e.g., a liquid crystalline liquid). The liquid crystalline fluid may be composed of a liquid crystal. In certain cases, the liquid crystal has a phase transition, such as a phase transition between an isotropic phase and a nematic phase (or vice versa). By “isotropic phase” or “isotropic” is meant a liquid crystal phase where the liquid crystals have no significant positional order or directional order. By “nematic phase” or “nematic” is meant a liquid crystal phase where the liquid crystals have no significant positional order, but have a detectable directional order. In some instances, the liquid crystal phase transition occurs in response to a stimulus applied to the liquid crystals. The stimulus may be any convenient stimulus that can induce a phase transition in the liquid crystals, such as, but not limited to, a change in temperature, an electrical stimulus, a magnetic stimulus, combinations thereof, and the like. In some cases, the stimulus that induces the phase transition in the liquid crystal is a change in temperature, e.g., heating or cooling. As such, the liquid crystalline fluid may be composed of a liquid crystal that has a temperature dependent phase transition. In some embodiments, the liquid crystalline fluid undergoes a phase transition from an isotropic phase to a nematic phase when the temperature of the liquid crystalline fluid is reduced to below the phase transition temperature. In some embodiments, the liquid crystalline fluid undergoes a phase transition from a nematic phase to an isotropic phase when the temperature of the liquid crystalline fluid is increased to above the phase transition temperature.

In certain embodiments, a temperature dependent liquid crystalline fluid has a phase transition temperature that is lower than the phase transition temperature of an organic ligand (or an organic ligand-functionalized nanoparticle) as described herein. As such, in some instances, the phase transition temperature (e.g., melting temperature or clearing point) of the organic ligand (or organic ligand-functionalized nanoparticle) is greater than the phase transition temperature of the liquid crystalline fluid. In certain instances, a temperature dependent liquid crystalline fluid has a phase transition temperature (e.g., for a phase transition between an isotropic phase and a nematic phase) ranging from 20° C. to 50° C., such as 25° C. to 45° C., or 30° C. to 40° C. In some cases, a temperature dependent liquid crystalline fluid has a phase transition temperature (e.g., for a phase transition between an isotropic phase and a nematic phase) of approximately 35° C., such as 34° C. to 36° C. Examples of liquid crystalline fluids that have a temperature dependent phase transition include, but are not limited to, 4-cyano-4′-pentylbiphenyl (5CB), and the like.

The nanoparticles may be dispersed in the mesomorphic material, such as liquid crystalline fluid, using any convenient method, such as, but not limited to, mixing, vortexing, shaking, applying sound energy (also referred to as “sonication” herein), combinations thereof, and the like. In some cases, the method includes applying sound energy to the nanoparticles in mesomorphic material to disperse the nanoparticles in the mesomorphic material. The nanoparticles may be dispersed in the mesomorphic material such that the nanoparticles are substantially evenly distributed throughout the mesomorphic material. For example, a mixture of the nanoparticles and mesomorphic material may be substantially homogeneous. In certain embodiments, the nanoparticles are dispersed in the mesomorphic material at room temperature (e.g., −25° C.). In other cases, the nanoparticles are dispersed in the mesomorphic material at a temperature other than room temperature, e.g., lower or higher than room temperature. In some instances, the nanoparticles are dispersed in the mesomorphic material at a temperature higher than room temperature. In certain embodiments, the nanoparticles are dispersed in the mesomorphic material at a temperature where the nanoparticles are present in a desired phase of the mesomorphic material, such as an isotropic phase or a nematic phase. For instance, embodiments of the methods include dispersing the nanoparticles in the mesomorphic material at a temperature where the nanoparticles are present in an isotropic phase of the mesomorphic material. In certain aspects, the temperature where the nanoparticles are present in an isotropic phase of the mesomorphic material is a temperature above the phase transition temperature of the mesomorphic material, such as a temperature ranging from 20° C. to 50° C., such as 25° C. to 45° C., or 30° C. to 40° C., such as a temperature of approximately 35° C., for example 34° C. to 36° C.

Embodiments of the method of producing the self-assembled microcapsules described herein also include inducing a phase transition in the mesomorphic material (e.g., the liquid crystalline liquid) to produce the self-assembled microcapsules. In certain embodiments, the phase transition of the mesomorphic material is a phase transition from an isotropic phase to a nematic phase. Thus, the method may include inducing a phase transition from an isotropic phase to a nematic phase in the mesomorphic material.

In some instances, inducing a phase transition in the mesomorphic material (e.g. a liquid crystalline liquid) is performed by applying a stimulus to the mesomorphic material. The stimulus may be any convenient stimulus that can induce a phase transition in the mesomorphic material, such as, but not limited to, a change in temperature, an electrical stimulus, a magnetic stimulus, combinations thereof, and the like. In some cases, inducing the phase transition in the mesomorphic material is accomplished by changing the temperature of the mesomorphic material, e.g., heating or cooling the mesomorphic material. In certain instances, inducing the phase transition in the mesomorphic material is accomplished by decreasing the temperature of the mesomorphic material to a temperature below the phase transition temperature of the mesomorphic material. Reducing the temperature of the mesomorphic material to a temperature below the phase transition temperature of the mesomorphic material may induce a phase transition of the mesomorphic material from an isotropic phase to a nematic phase. In some cases, at the isotropic to nematic phase transition in a homogeneous mesomorphic material, domains of nematic ordering form and grow as the mesomorphic material is cooled through the transition temperature. As the nematic domains form and increase in size, isotropic domains began decreasing in size. In some instances, the dispersed nanoparticles (e.g., organic ligand-functionalized nanoparticles) in the mesomorphic material may preferentially locate in the shrinking isotropic domains. As the nanoparticles aggregate at the interface between the isotropic and nematic domains or mesomorphic state, the nanoparticles may form a microcapsule of stably associated nanoparticles as described herein. For example, a microcapsule may be produced, such as a shell configuration having a spherical surface. Without being bound to any particular theory, by controlling the phase transition process rate can control the size of the microcapsule being formed. In certain instances, slowing the cooling rate of the mesomorphic material (e.g. a liquid crystalline liquid) results in larger microcapsules being formed.

### Methods

Aspects of the present disclosure include methods of delivering one or more substrates to an individual, where the substrate is released from a self-assembled microcapsule described herein by activation with a power source, wherein the maximum temperature change at the microcapsule surface upon activation with the power source is 75° C. or less. The method of delivering one or more substrates to an individual includes administering an effective amount of a self-assembled microcapsule composed of organic ligand functionalized nanoparticles, containing one or more substrates encapsulated inside the microcapsule, and applying activation from a power source to release the one or more substrates. The self-assembled microcapsule used in the method may be composed of any organic ligand functionalized nanoparticles as described herein. For instance, the nanoparticles may be functionalized with an organic ligand having a structure of formula (I) as described herein.

In certain embodiments of the methods of delivering one or more substrates to an individual, the self-assembled microcapsule may be composed of nanoparticles substituted with an organic ligand selected from (L1)-(L16) as described herein. In some embodiments of the methods described herein, the nanoparticles are substituted with an organic ligand according to formula (I) as described herein. In some embodiments of the methods of delivering one or more substrates to an individual, the self-assembled microcapsule may be composed of gold nanoparticles substituted with an organic ligand as described herein. In certain embodiments of the methods, the nanoparticles are densely packed so as to prevent leakage of the encapsulated substrate over a period of several months and allow rapid release of the substrate when activated with a power source. In some cases, the mean inter-particle separation of the nanoparticles in the microcapsule wall is about 12 nm, such as 12.1 nm, 12.2 nm, 12.3 nm, 12.4 nm or 12.5 nm. In some cases, the mean inter-particle separation of the nanoparticles in the microcapsule wall is less than 12 nm, such as 11 nm, 11.1 nm, 11.2 nm, 11.3 nm, 11.4 nm, 11.5 nm, 11.6 nm, 11.7 nm, 11.8 nm or 11.9 nm.

Aspects of the methods include activation from a power source to release the one or more substrates encapsulated within the self-assembled microcapsules as described herein. In some embodiments, activation is achieved with a low power source. In some cases, the power source is below the American National Standards Institute (ANSI) maximum permissible exposure limit. In certain cases the maximum permissible exposure power density of the power source is 100 mW/cm\hat{ }2 or less, such as 75 mW/cm\hat{ }2 or less, 50 mW/cm\hat{ }2 or less, 25 mW/cm\hat{ }2 or less, 10 mW/cm\hat{ }2 or less, or 5 mW/cm\hat{ }2 or less. In some cases, the maximum permissible exposure time to the power source is 6 minutes or less, such as 5 minutes or less, 4 minutes or less, 3 minutes or less, 2 minutes or less, or 1 minute or less.

In some embodiments of the methods, release of the substrate is activated through localized surface plasmon resonance (LSPR) stimuli.

In some cases, the release of the substrate is activated at an excitation wavelength of from 200 nm to 1 mm, such as from 300 nm to 1 mm, from 400 nm to 1 mm, from 500 nm to 1 mm, from 600 nm to 1 mm, from 700 nm to 1 mm, from 800 nm to 1 mm, from 900 nm to 1 mm, from 1000 nm to 1 mm, from 1200 nm to 1 mm, from 1400 nm to 1 mm, from 1600 nm to 1 mm, from 1800 nm to 1 mm, or from 3 μm to 1 mm. In certain cases, the excitation wavelength is from 400 nm to 1 mm, such as 450 nm to 1 mm, 550 nm to 1 mm, 650 nm to 1 mm, 750 nm to 1 mm, 850 nm to 1 mm, or 950 nm to 1 mm. In some embodiments, the method includes applying ultraviolet (UV) radiation to activate release of the substrate. For example, the method may include applying UV radiation having a wavelength ranging from 100 nm to 400 nm, such as 150 nm to 400 nm, or 200 nm to 400 nm, or 300 nm to 400 nm. In some embodiments, the method includes applying visible (VIS) radiation to activate release of the substrate. For example, the method may include applying VIS radiation having a wavelength ranging from 400 nm to 800 nm, such as 500 nm to 800 nm, or 600 nm to 800 nm, or 700 nm to 800 nm. In some embodiments, the method includes applying infrared (IR) radiation to activate release of the substrate. For example, the method may include applying IR light having a wavelength ranging from 800 nm to 1400 nm, or 1400 nm to 3 μm, or 3 μm to 1 mm. In certain instances, release of the substrate is activated at 514 nm. In some instances, release of the substrate is activated at 488 nm or 561 nm.

In certain cases, release of the substrate is activated with a light that has a wavelength from 200 nm to 1 mm at a power density of 100 mW/cm\hat{ }2 or less. In some cases, release of the substrate is activated using 2 mW or less of incident power and full release is obtained within 1-5 seconds. For example, 2 mW or incident power and full release of the substrate in 1.2 s.

In certain embodiments full release of the substrate is obtained in 6 minutes or less from the time of activation with a power source, such as 5 minutes or less, 4 minutes or less, 3 minutes or less, 2 minutes or less, or 1 minute or less.

In some instances of the methods disclosed herein, during activation of the self-assembled microcapsules with an optical power source localized heating of the shell surface is observed, causing release of the substrates encapsulated within. Without being bound to any particular theory, the microcapsules may break open due to increased thermal fluctuations of the organic ligands attached to the nanoparticles weakening the shell wall.

In one embodiment, the maximum temperature change at the microcapsule surface upon activation with a power source is 75° C. or less. In some cases, the maximum temperature change at the microcapsule surface upon activation with a power source is less than 75° C., such as less than 70° C., less than 65° C., less than 60° C., less than 55° C., less than 50° C., or even less. In some cases, the temperature change at the microcapsule surface upon activation with a power source is 50° C. or less. In some cases, the maximum temperature change at the microcapsule surface upon activation with a power source is less than 50° C., such as less than 45° C., less than 40° C., less than 35° C., less than 30° C., or even less.

### Utility

The self-assembled microcapsules of the present disclosure find use in applications such as the encapsulation of active agents and live cells and release of the encapsulated substrates upon activation with a power source. Encapsulation of the active agent inside the microcapsule may facilitate one or more of: delivery of the active agent to a desired site; formulation of the active agent into a desired formulation; increased stability of the active agent; controlled release of the active agent; delayed release of the active agent; and the like. The self-assembled microcapsules of the present disclosure find particular use in targeted controlled release of its encapsulated cargo to a living host, such that at the site of release in the living host the surrounding healthy tissue is not damaged. The self-assembled microcapsules of the present disclosure also find use in targeted controlled release of its encapsulated cargo at any targeted location, such as at a site of release in a living host, or an environment that does not include a living host, such as a chemical solution.

### Kits

Aspects of the present disclosure additionally include kits that include self-assembled microcapsules as described in detail herein. In some instances, the kit includes a packaging for containing the self-assembled microcapsules. In certain embodiments, the packaging may be a sealed packaging, e.g., in a water vapor-resistant container, optionally under an air-tight and/or vacuum seal. In certain instances, the packaging is a sterile packaging, configured to maintain the microcapsules enclosed in the packaging in a sterile environment. By “sterile” is meant that there are substantially no microbes (such as fungi, bacteria, viruses, spore forms, etc.). The kits may further include a fluid (e.g., a liquid). For instance, the kit may include a liquid, such as a liquid in which the microcapsule structures are provided. For example, the microcapsule may be dispersed in the liquid. Liquids in which the microcapsules may be dispersed include, but are not limited to, a mesomorphic material (e.g., liquid crystalline liquid), a solvent (e.g., a pharmaceutically acceptable organic solvent), and the like.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., CD, DVD, Blu-Ray, computer-readable memory (e.g., flash memory), etc., on which the information has been recorded or stored. Yet another means that may be present is a website address which may be used via the Internet to access the information at a removed site. Any convenient means may be present in the kits.

As can be appreciated from the disclosure provided above, embodiments of the present invention have a wide variety of applications. Accordingly, the examples presented herein are offered for illustration purposes and are not intended to be construed as a limitation on the invention in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by mass, molecular mass is mass average molecular mass, temperature is in degrees Celsius, and pressure is at or near atmospheric.

### EXAMPLES

### Example 1—Microcapsule Formation

The organic ligands used in this example were L1-L16. The ligand molecules contained a rigid aromatic core region surrounded by flexible domains at the termini, which gave the ligand the ability to interact with the anisotropic host phase to produce uniform dispersion on the nanoparticle surface. The flexible alkyl amine or alkyl thiol side-arm segment of the organic ligands bound to the nanoparticle surface in the ligand exchange process. Ligands were attached (also referred to herein as “exchanged”) on to the surface of commercial gold nanoparticles (AuNPs) (Nano Partz Inc.). The AuNPs dissolved in chloroform were precipitated by washing with 2:1 methanol:chloroform mixture. The mixture was centrifuged for 30 min and the supernatant was discarded. The precipitate was then re-dissolved in chloroform and washed two more times with a 2:1 methanol:chloroform mixture. The precipitate was then dissolved in chloroform and a solution of the organic ligands dissolved in toluene (40 mM). The AuNP/organic ligand mixture was then heated to 45° C. and stirred for five hours. The mixture was then left to cool back to room temperature. Ethyl acetate was then added to the ligand-exchange gold solution and centrifuged. The precipitate was washed two more times using a 1:1:2 solution of toluene, chloroform, and methanol. The precipitate was finally suspended in chloroform.

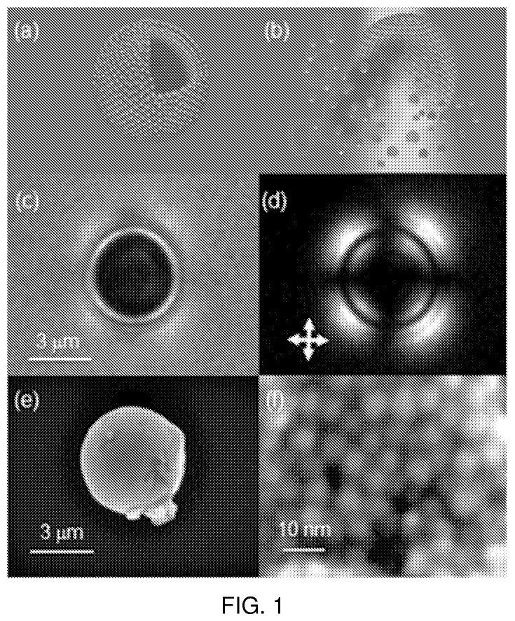

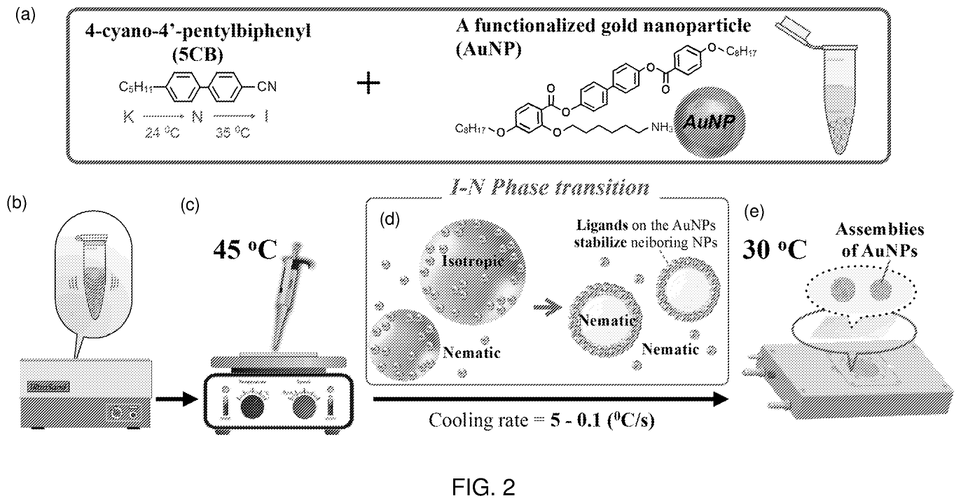

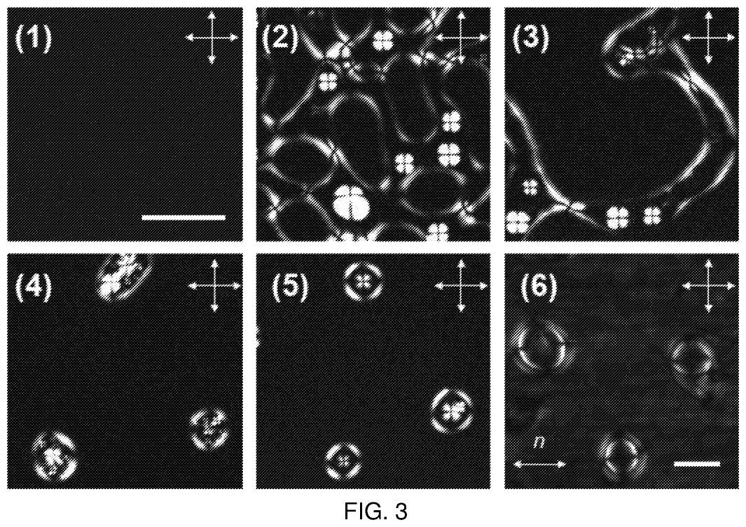

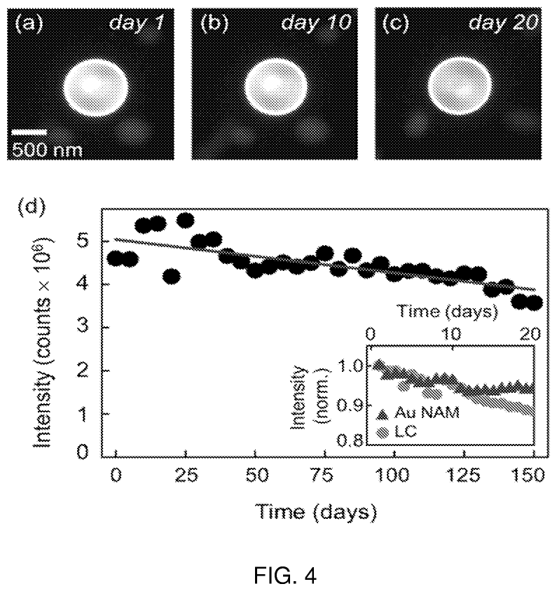

The organic ligand functionalized AuNPs suspended in chloroform (1.2% (wt)) were then dispersed into the common rod-like nematic liquid crystal (LC), 4-cyano-4′-pentylbiphenyl (5CB) (FIG. 2, panel a) which exhibited a nematic-to-isotropic transition at 35.5° C. The LC-AuNPs mixture was then sonicated in a 40° C. bath for 5 h in an Eppendorf tube with the cap removed, allowing the LC to remain in the isotropic phase while the solvent (chloroform) in which the AuNPs were suspended during functionalization gradually evaporated from the LC (FIG. 2, panel b). Solvent removal was verified by measuring the nematic-to-isotropic phase-transition point using a Perkin-Elmer differential scanning calorimeter (DSC). After achieving a homogenous dispersion, the mixture was deposited onto a microscope slide mounted on a temperature-controlled Linkham LTS350 hot-stage and sandwiched with a cover slip (FIG. 2, panel c). Slide and cover clip surfaces were pre-treated with cetyltrimetylammonium bromide (CTAB) to encourage homeotropic alignment of the LC molecules. When homogeneous dispersion was observed, the microscope slide was transported to another temperature-controlled Linkham LTS350 hot-stage (33-30° C., FIG. 2, panel e). As the LC-AuNP mixture was cooled into the nematic phase (cooling rate 5 to 0.1° C./s), it separated into LC-AuNP-rich droplets and an LC-rich phase (FIG. 2, panel d and FIG. 3). During this segregation, the functionalized AuNPs moved into the shrinking isotropic domains and layer-by-layer spherical wall formation of organic ligand functionalized AuNPs microcapsule was observed (FIG. 1, panel e and FIG. 4, panel f). Microcapsule size was controlled by the cooling rate. Fast cooling (3° C./s) resulted in the formation of shells with diameter ˜1 μm, while a slower rate (0.2° C./s) produced shells as large as 5 μm.

Polarized optical microscopy was used to track the LC texture of a homeotropically aligned LC cell throughout the transition from isotropic (40° C.) into the nematic phase (30° C.), thus demonstrating the AuNP microcapsule formation process indirectly (FIG. 3, panels 1-5). With reference to FIG. 3, sequence of images (1)-(5), the polarizers are crossed as indicated by the white arrows and the birefringence from the microcapsule structures can be seen with characteristic nematic extinction crosses clearly visible on the images. A white scale bar represents 3 μm. In FIG. 3, panel 6, the host nematic phase appeared dark because the nematic director, n, was oriented parallel with one of the crossed polarizer directions (indicated by the white arrows). This image shows the birefringence of the microcapsule interior and the topological defects surrounding the microshell. Colloidal particles with a radial ligand distribution producing either homeotropic or planar surface anchoring conditions created characteristic topological defects when surrounded by the nematic phase. Such defects were visualized with polarized optical microscopy, providing information on ligand organization at the particle surface. The presence of horizontal Saturn ring defects around some shells in FIG. 3, panel 6, indicated that the outer ligands were aligned parallel to the microcapsule surface, producing a planar surface anchoring condition. In addition, bipolar-type defects were also observed as expected for this geometry. In this image, the surrounding 5CB material was aligned using a standard rubbed polyvinyl alcohol (PVA) alignment layer for optimal defect visualization.

Three-dimensional structures (e.g., microcapsule structures as described herein) using AuNP exchanged with ligands L1-L16, respectively, were prepared according to the procedures described herein.

### Structural Characterization

Using scanning electron microscopy, (SEM), shells post-extraction from the host LC were observed (FIG. 4, panel b), which led to a slight deformation. Higher resolution SEM showed a densely-packed arrangement of the individual AuNPs in the shell wall (FIG. 4, panel b, enlarged image), and small angle X-ray scattering (SAXS) indicated that the mean inter-particle separation of the AuNPs in the wall was 12.1 nm. This densely-packed configuration allowed the microcapsule to create plasmonic heat with low laser power.

### Example 2—Encapsulation Procedure

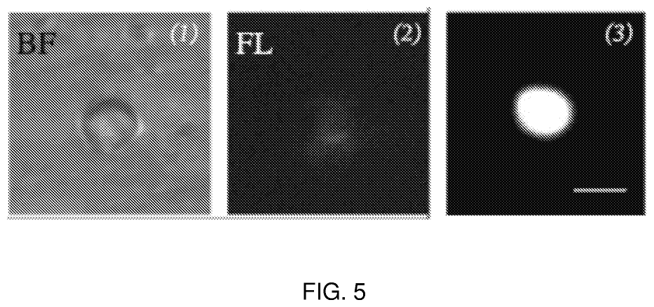

For encapsulation within the organic ligand functionalized AuNPs, Lumogen F Red 300 (BASF) was used, a dye with a high quantum yield. The dye was dissolved in toluene and added to a LC-AuNP mixture (1.2 wt %), to obtain a composite with a concentration of 2 mM. Alternatively, fluorescent beads were used (0.2 μm carboxylate coupling surface fluorescent spheres with emission of 515 nm). The fluorescent beads were diluted in deionized water (1:1000), then added to a LC-AuNP mixture to obtain a composite with a dilution of 1:5000. Following sonication and shell formation (as per the procedure outlined under example 1), The resulting microcapsules were spun down with a centrifuge (8000 rpm for 30 min), the supernatant discarded, and the precipitate dye-containing microcapsules were re-suspended in dye-free LC (5CB). The process was repeated five times until a clear contrast between the shells containing Lumogen F Red and the host LC was observed (FIG. 4, panels a-c). The sample was imaged using epifluorescence microscopy after each centrifugation step to ensure that the shells remained undamaged. To quantify the stability of encapsulation in the functionalized AuNPs, the Lumogen F Red fluorescence intensity was measured, as a proxy for the dye concentration in the AuNPs, over 5 months. At each time, the dye containing microcapsules were deposited onto a glass slide, overlaid with a cover slip, and the edges sealed with wax to minimize changes in the sample due to the diffusion of oxygen and water. Visually, a small decrease in the emission with time, ˜20% over 150 days was observed (FIG. 4, panels a-c), and the spatially integrated fluorescence intensity exhibited linear quenching (FIG. 5, panel d). A control with Lumogen F Red dispersed in LC (5CB) and imaged under identical conditions for 10 days demonstrated that photobleaching was a small but significant factor in quantifying the evolution of intensity of Lumogen F Red in the experiments (FIG. 4, panel d, inset). In fact, after 10 days, it was observed that encapsulation in the functionalized AuNP microcapsules reduced the effects of photo bleaching compared to the dye in the bulk LC in the control. It was therefore concluded that Lumogen F Red remained stably encapsulated in the AuNP microcapsules for at least 5 months with minimal to no leakage.

Having confirmed that the invention microcapsules stably encapsulate a substrate for substantial periods of time, the organic ligand functionalized AuNPs ability to encapsulate green fluorescent protein (GFP) labeled E. coli was investigated.

GFP labeled E. coli LB agar plates were incubated overnight at 37° C., then incubated with LB media culture at 37° C. for 24 hours in a shaking incubator. After the cultures were grown, they were subcultured (1:500) into fresh LB agar plates, 0.5 mL of stational or exponential-phase cultures were pelleted (5,000×g for 2 min at room temperature), and the pellets were re-suspended in 0.1 mL fresh LB media. The E. coli-LB media solution (1 μL) was then added to a LC-AuNP mixture (20 μL) and the resulting mixture was sonicated for 15 minutes at 40° C. Following sonication and shell formation (as per the procedure outlined under example 1), the resulting microcapsules were spun down with a centrifuge (8000 rpm for 30 min), the supernatant (un-captured E. coli) discarded, and the E. coli-containing microcapsules were re-suspended in LC (5CB). The process was repeated at least two times until a clear contrast between the shells containing E. coli and the host LC was observed. The sample was imaged using a fluorescence microscope after each centrifugation step to ensure that the shells remained undamaged (FIG. 5, panels 1-3.

FIG. 5 shows the encapsulation of fluorescence beads under the fluorescence microscope. Panel 1 exhibits a Bright field microscopy (BF) image; Panel 2 exhibits a fluorescence (FL) image of GFP labeled E. coli bacterium captured within a microcapsule. Panel 3 exhibits a 0.2 μm diameter fluorescent spheres in a AuNP microcapsule visualized in bright under a fluorescence microscope. The white scale bar represents 2 μm.

### Example 3—Microcapsule Breaking Procedure

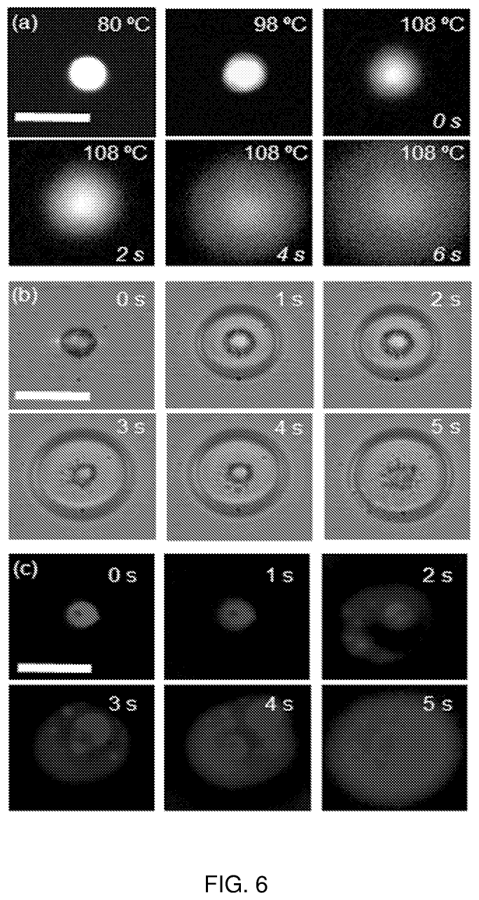

Images of microcapsule breaking were performed on a TIRF microscope built with a Ti-E Eclipse stand (Nikon Instruments). The objective used was an Apo TIRF 100× (N.A. 1.49). CUBE diode 405 nm laser (Coherent), Sapphire OPSL 488 nm, 514 nm, and 561 nm lasers (Coherent), and OBIS 647 nm (Coherent) were combined into a fiber optic cable and into a TIRF illuminator (Nikon) attached to the microscope stand. Shuttering of the laser illumination was controlled by an acousto-optic tunable filter (AA Optoelectronics) before the fiber coupler. Images were acquired with an iXon3+ 887 EMCCD (Andor Technology) camera and synchronization between components was achieved using pManager [A] with a microcontroller (Arduino). The microshell breaking in FIG. 6, panels a-c, was observed by exposing microscope LC-AuNP microcapsule slides to each excitation (405, 488, 514, 561, 647 nm) with incident laser power at 0.5, 0.75, 1, 1.5, and 2 mW in a temperature controlled chamber (HaisonTech) at 29° C.

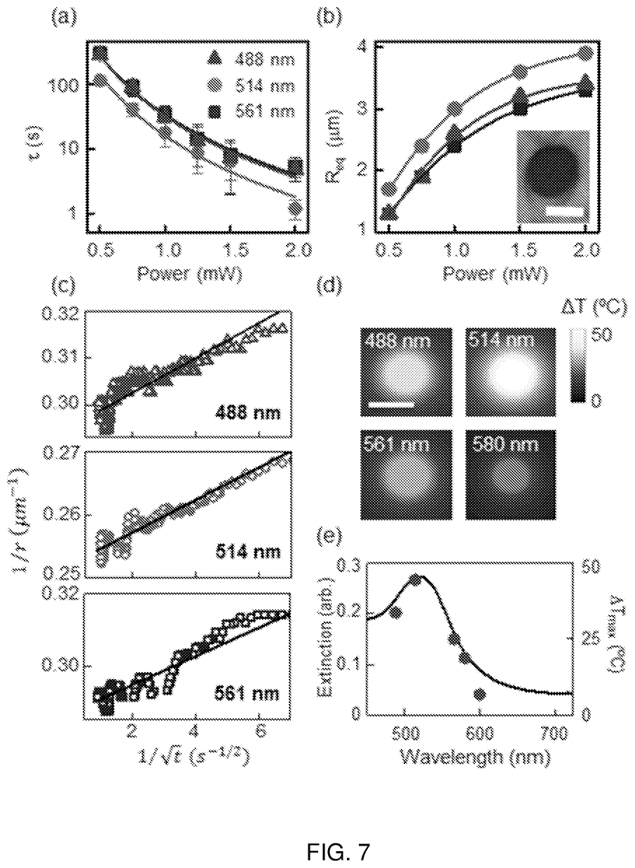

To explore the thermal stability of the microcapsules, the fluorescence of a microcapsule containing Lumogen F Red was monitored while the sample was heated on a temperature-controlled Linkham stage at 1° C./min, beginning at 25° C., shown in FIG. 7, panels a-e. The microcapsule structure remained unchanged up to a temperature of 98° C. At 98° C., the shell began to deform slightly. At 108° C., the rigid shell disintegrated and the dye leaked out, with the structure completely collapsing within 6 s (FIG. 7, panel a). Having confirmed that the microcapsules could be destroyed through thermal perturbations and their enclosed cargo released, it was sought to trigger release though LSPR-mediated stimuli. The microcapsules containing Lumogen F Red were illuminated with a 488-nm laser at a power of 2 mW at room temperature, and a series of bright-field images were acquired at an interval of 5 s to monitor the structure of the microcapsule (FIG. 7, panel b). After approximately 3 s of illumination, the shell began to disintegrate, and by 5 s it had collapsed completely. Simultaneous fluorescence imaging confirmed that the encapsulated dye leaked out within 2 s of illumination (FIG. 6, panel c), and was released entirely after 5 s.

The release time, T, was characterized as a function of incident power for different excitation wavelengths (FIG. 7). The fastest response was observed for excitation at 514 nm, which is closest to the LSPR (520 nm). For the highest incident power (2 mW), release was extremely rapid (T=1.2 s). More importantly, release within 2 min was achieved with power as low as 0.5 mW. Furthermore, the microcapsules responded to slightly off-resonant light (488 and 561 nm) almost as well, with T ranging from 6 s to 5 min with varying incident power, increasing the responsive spectral band to ±40 nm around the LSPR. Tuning the excitation wavelength farther away failed to elicit any response even at higher powers. This spectral selectivity indicated that targeting of specific microcapsules based on the size of AuNPs constituting the shell wall may be achieved.

In the time-lapse imaging of shell disintegration (FIG. 6, panel b) it was observed that after 1 s a bubble had formed around the microcapsule. Polarized optical microscopy (POM) images indicated that the temperature increase on and near the microcapsule surface caused a transition in the surrounding LC from the nematic to isotropic phase, which was what constituted the bubble (FIG. 7, panel b, inset). The LC outside remained in the nematic phase, with the bubble radius demarcating the phase boundary.

### Example 4—Synthesis of Organic Ligands

Some exemplary organic ligands described herein were synthesized according to procedures published in: Rodarte, A. L. et al. Self-assembled nanoparticle micro-shells templated by liquid crystal sorting. Soft Matt. 11, 1701-1707 (2015).

Although the foregoing embodiments have been described in some detail by way of illustration and example for purposes of clarity of understanding, it is readily apparent to those of ordinary skill in the art in light of the teachings of the present disclosure that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

Notwithstanding the appended claims, the disclosure set forth herein is also described by the following clauses.

Clause 1. A self-assembled microcapsule comprising organic ligand-functionalized nanoparticles and one or more substrates encapsulated inside the microcapsule, wherein the microcapsule releases the substrate upon activation with a power source and the maximum temperature change at the microcapsule surface upon activation with the power source is 75° C. or less.
Clause 2. The self-assembled microcapsule of clause 1, wherein the organic ligand has the structure of formula (I):

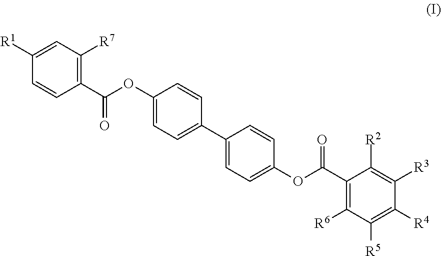

wherein

R¹ and R⁷ are each independently selected from, C₁-C₈ alkoxy, and C₁-C₈ alkoxy substituted with an amine or thiol group; and

R², R³, R⁴, R⁵ and R⁶ are each independently selected from H, halogen, hydroxyl, azido, alkyl, substituted alkyl, alkenyl, substituted alkenyl, alkynyl, substituted alkynyl, C₁-C₁₂ alkoxy, substituted alkoxy, amino, substituted amino, cycloalkyl, substituted cycloalkyl, heterocycloalkyl, substituted heterocycloalkyl, aryl, substituted aryl, heteroaryl, substituted heteroaryl, phosphate, substituted phosphate, phosphoryl, substituted phosphoryl, thiol, substituted thiol or combinations thereof.

Clause 3. The self-assembled microcapsule of clause 1 or clause 2, wherein the mean inter-particle separation of the nanoparticles is from 1 nm to 100 nm.
Clause 4. The self-assembled microcapsule of any one of clauses 1 to 3, wherein the nanoparticles are composed of upconversion nanoparticles, plasmonic nanoparticles, or combinations thereof.
Clause 5. The self-assembled microcapsule of any one of clauses 1 to 4, wherein the nanoparticles are composed of a material selected from a semiconductor material, a metal, a metal oxide, a metalloid, a metal coated material, an oxide, a magnetic material, a nanosome, a lipidsome and a polymer, or combinations thereof.
Clause 6. The self-assembled microcapsule of clause 5, wherein the nanoparticles are composed of gold nanoparticles, silver nanoparticles, zinc oxide nanoparticles, gold coated nanoparticles, silver coated nanoparticles, zinc coated nanoparticles or combinations thereof.
Clause 7. The self-assembled microcapsule of clause 5, wherein the nanoparticles are composed of iron oxide nanoparticles, cobalt nanoparticles, graphene coated iron oxide nanoparticles, graphene coated cobalt, silica coated iron oxide and silica coated cobalt or combinations thereof.
Clause 8. The self-assembled microcapsule of clause 6, wherein the nanoparticles are composed of gold nanoparticles.
Clause 9. The self-assembled microcapsule of any one of clauses 1 to 8, wherein the microcapsule has a spherical surface.
Clause 10. The self-assembled microcapsule of any one of clauses 1 to 9, wherein the organic ligand is selected from the group consisting of:

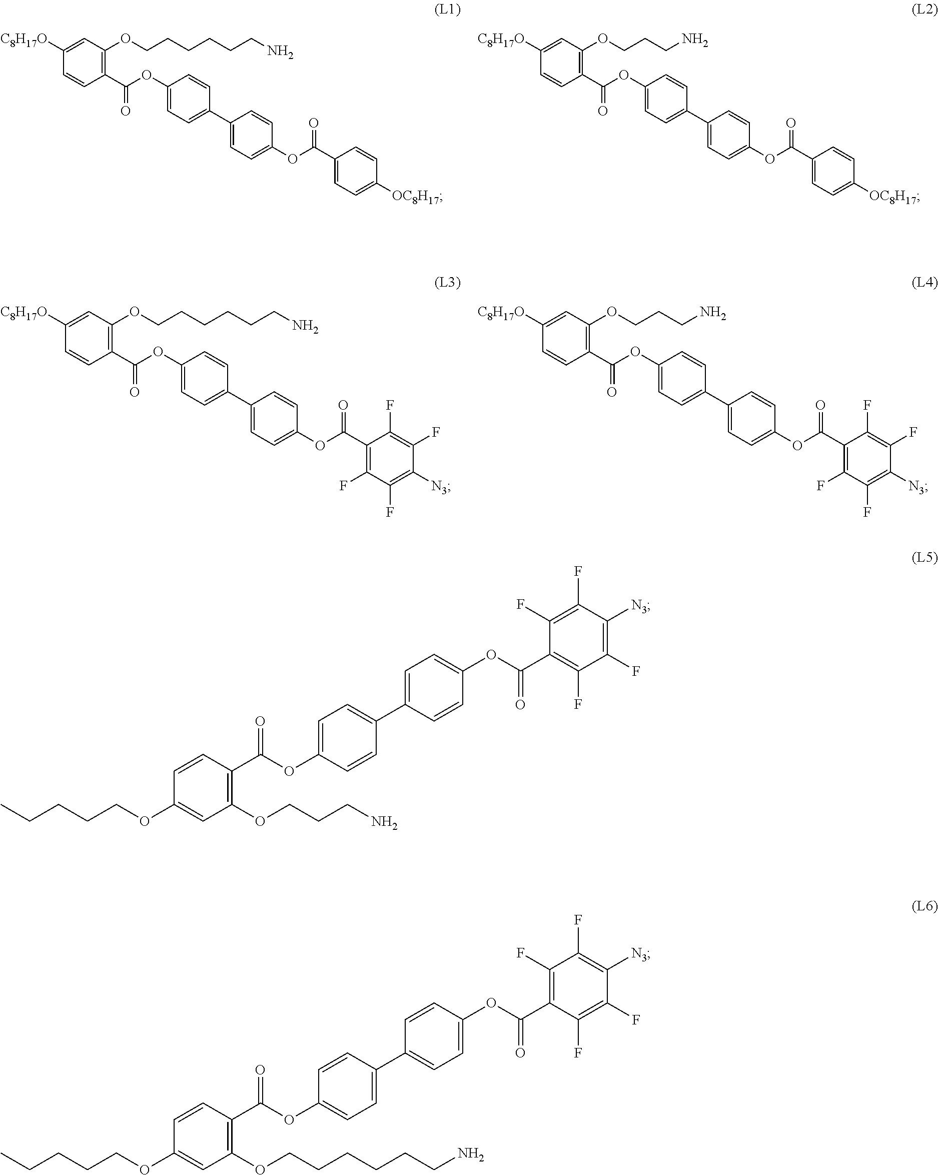

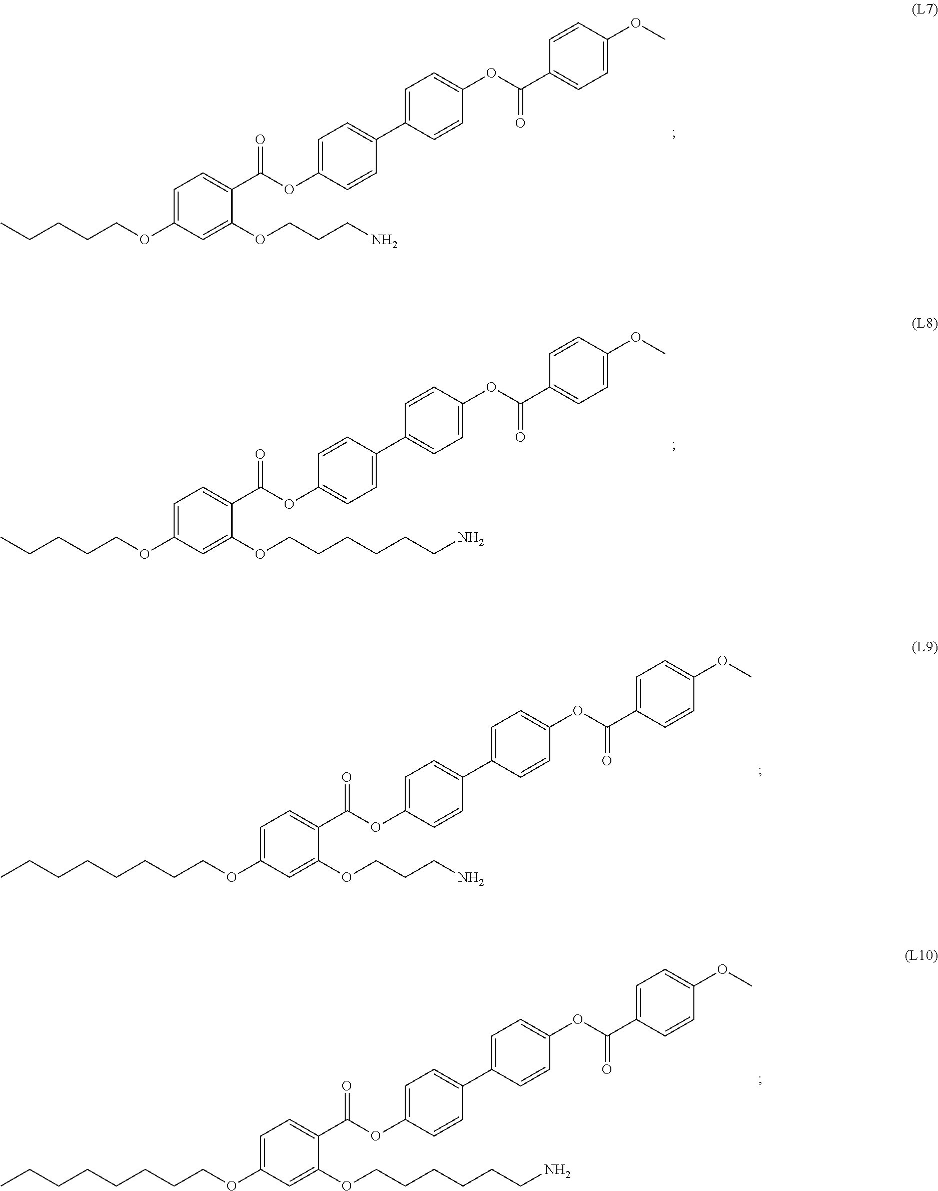

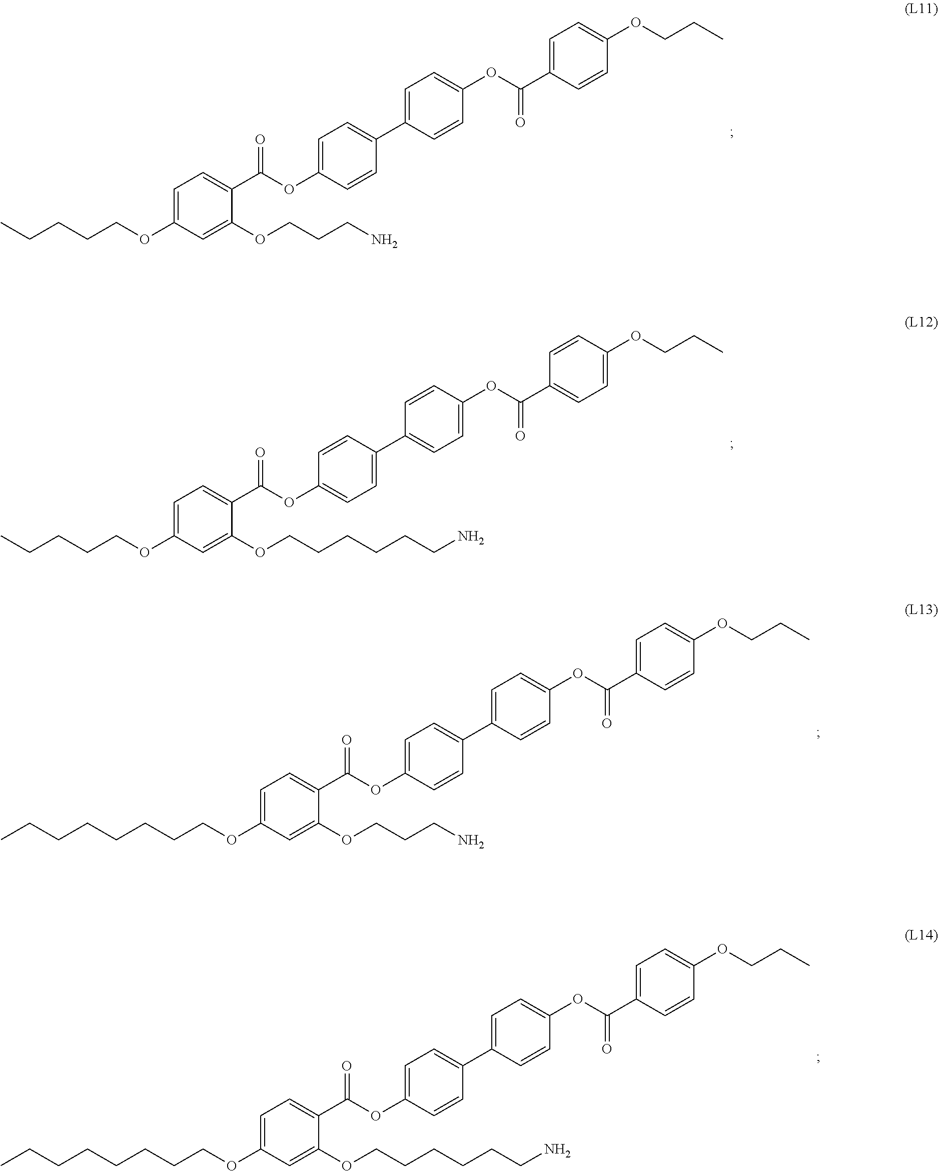

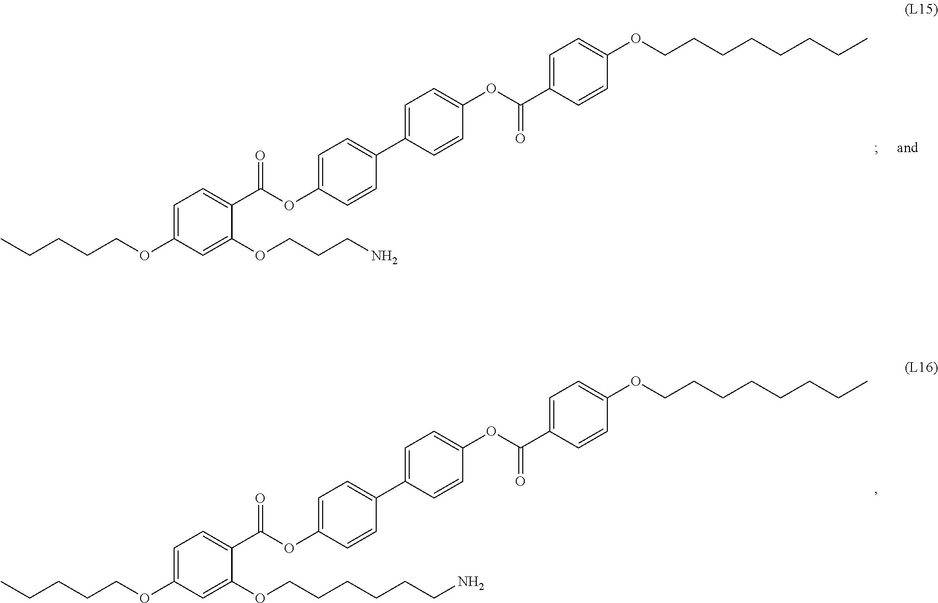

or combinations thereof.

Clause 11. The self-assembled microcapsule of clause 9, wherein the microcapsule has an average diameter of 100 nm to 100 μm.
Clause 12. The self-assembled microcapsule of clause 9, wherein the nanoparticles have an average diameter of 1 nm to 100 nm.
Clause 13. The self-assembled microcapsule of any one of clauses 1 to 12, wherein the microcapsule has a thickness of from 1% to 50% of the volume of the microcapsule.
Clause 14. The self-assembled microcapsule of any one of clauses 1 to 13, wherein the substrate is an active agent.
Clause 15. The self-assembled microcapsule of any one of clauses 1 to 13, wherein the substrate is live cells.
Clause 16. The self-assembled microcapsule of any one of clauses 1 to 15, wherein the power source is below the American National Standards Institute (ANSI) maximum permissible exposure limit.
Clause 17. The self-assembled microcapsule of any one of clauses 1 to 16, wherein the maximum permissible exposure power density is 100 mW/cm\hat{ }2 or less.
Clause 18. The self-assembled microcapsule of any one of clauses 1 to 17, wherein the maximum permissible exposure time to the power source is 6 minutes or less.
Clause 19. The self-assembled microcapsule of any one of clauses 1 to 18, wherein the release of the substrate is activated through localized surface plasmon resonance (LSPR) stimuli.
Clause 20. The self-assembled microcapsule of any one of clauses 1 to 19, wherein the release of the substrate is activated at an excitation wavelength of from 200 nm to 1 mm.
Clause 21. The self-assembled microcapsule of any one of clauses 1 to 19, wherein the release of the substrate is activated at an excitation wavelength of from 400 nm to 1 mm.
Clause 22. The self-assembled microcapsule of any one of clauses 1 to 21, wherein the release of the substrate is activated with a light that has a wavelength from 200 nm to 1 mm at a power density 100 mW/cm\hat{ }2 or less.
Clause 23. The self-assembled microcapsule of any one of clauses 1 to 22, wherein full release of the substrate is obtained in 6 minutes or less from the time of activation with a power source.
Clause 24. The self-assembled microcapsule of any one of clauses 1 to 23, wherein the temperature change at the microcapsule surface upon activation with a power source is 50° C. or less.
Clause 25. A composition comprising:

a liquid; and

a self-assembled microcapsule of any one of clauses 1 to 24 in the liquid.

Clause 26. The composition of clause 25, wherein the liquid is a pharmaceutically acceptable liquid or a mesomorphic material.
Clause 27. A method of delivering one or more substrates to an individual, the method comprising:

administering an effective amount of a self-assembled microcapsule comprising, organic ligand-functionalized nanoparticles and one or more substrates encapsulated inside the microcapsule, to an individual; and

applying activation from a power source to release the one or more substrates, wherein the maximum temperature change at the microcapsule surface upon activation with the power source is 75° C. or less.

Clause 28. The method of clause 27, wherein the organic ligand has the structure of formula (I):

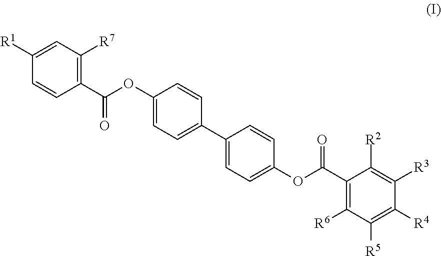

wherein

R¹ and R⁷ are each independently selected from, C₁-C₈ alkoxy, and C₁-C₈ alkoxy substituted with an amine or thiol group; and

R², R³, R⁴, R⁵ and R⁶ are each independently selected from H, halogen, hydroxyl, azido, alkyl, substituted alkyl, alkenyl, substituted alkenyl, alkynyl, substituted alkynyl, C₁-C₁₂ alkoxy, substituted alkoxy, amino, substituted amino, cycloalkyl, substituted cycloalkyl, heterocycloalkyl, substituted heterocycloalkyl, aryl, substituted aryl, heteroaryl, substituted heteroaryl, phosphate, substituted phosphate, phosphoryl, substituted phosphoryl, thiol and substituted thiol and combinations thereof.

Clause 29. The method of clause 27 or 28, wherein the organic ligand is selected from the group consisting of:

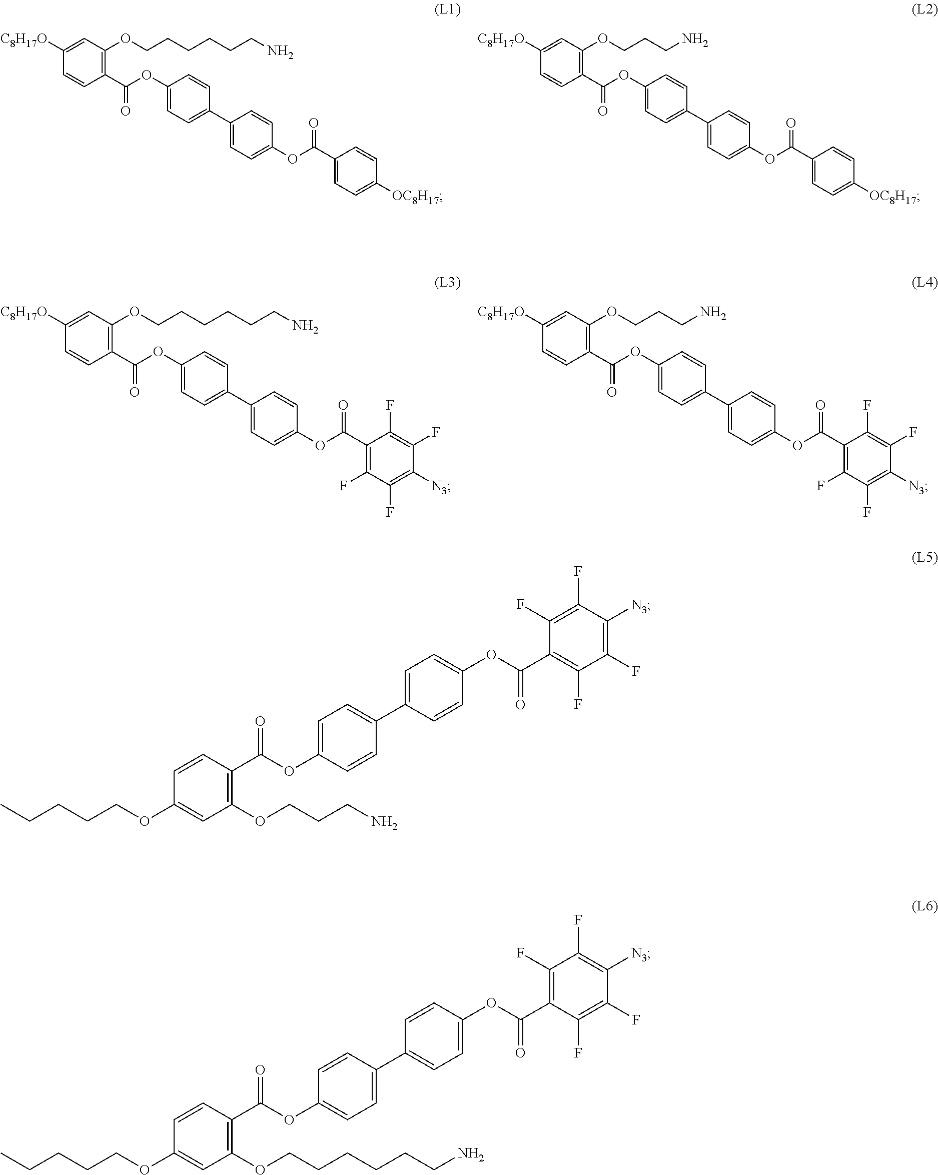

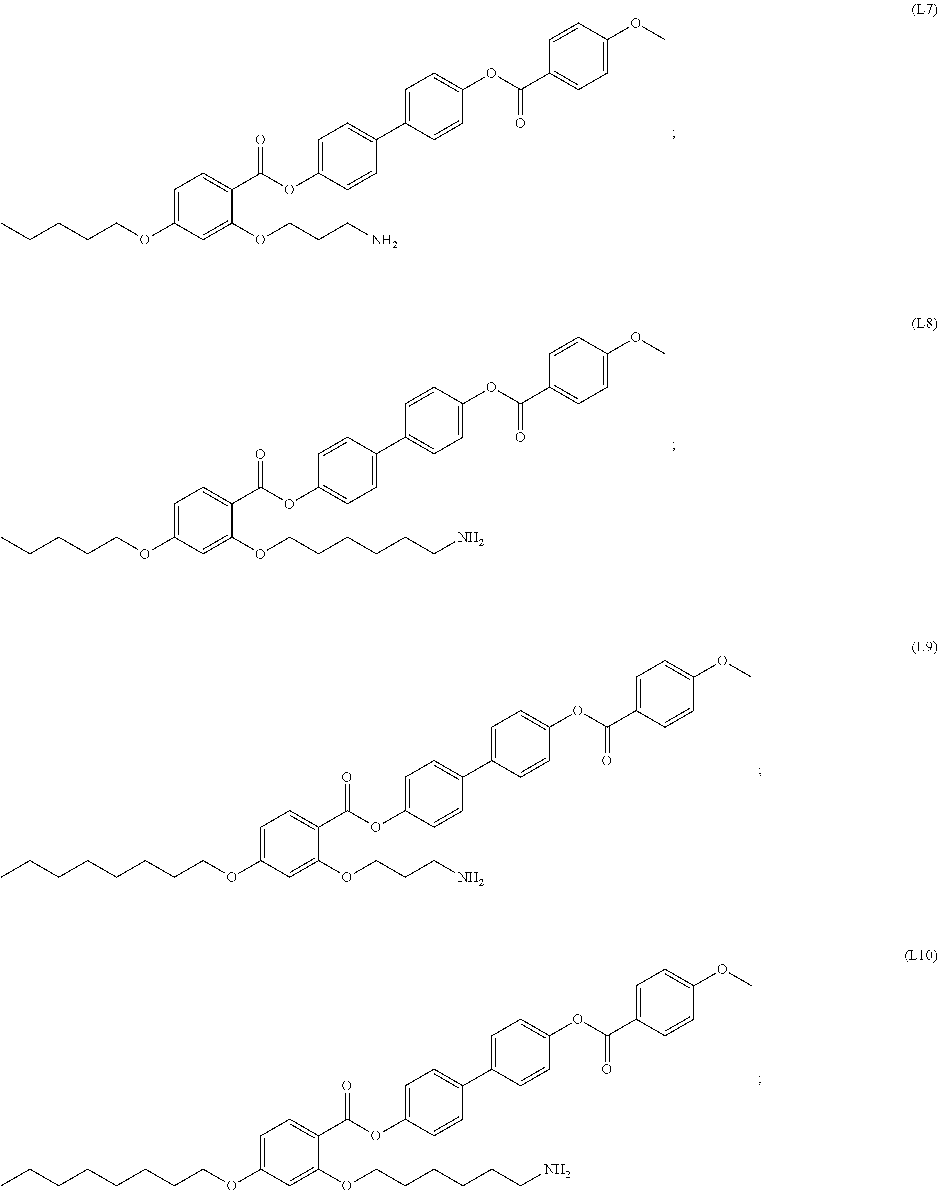

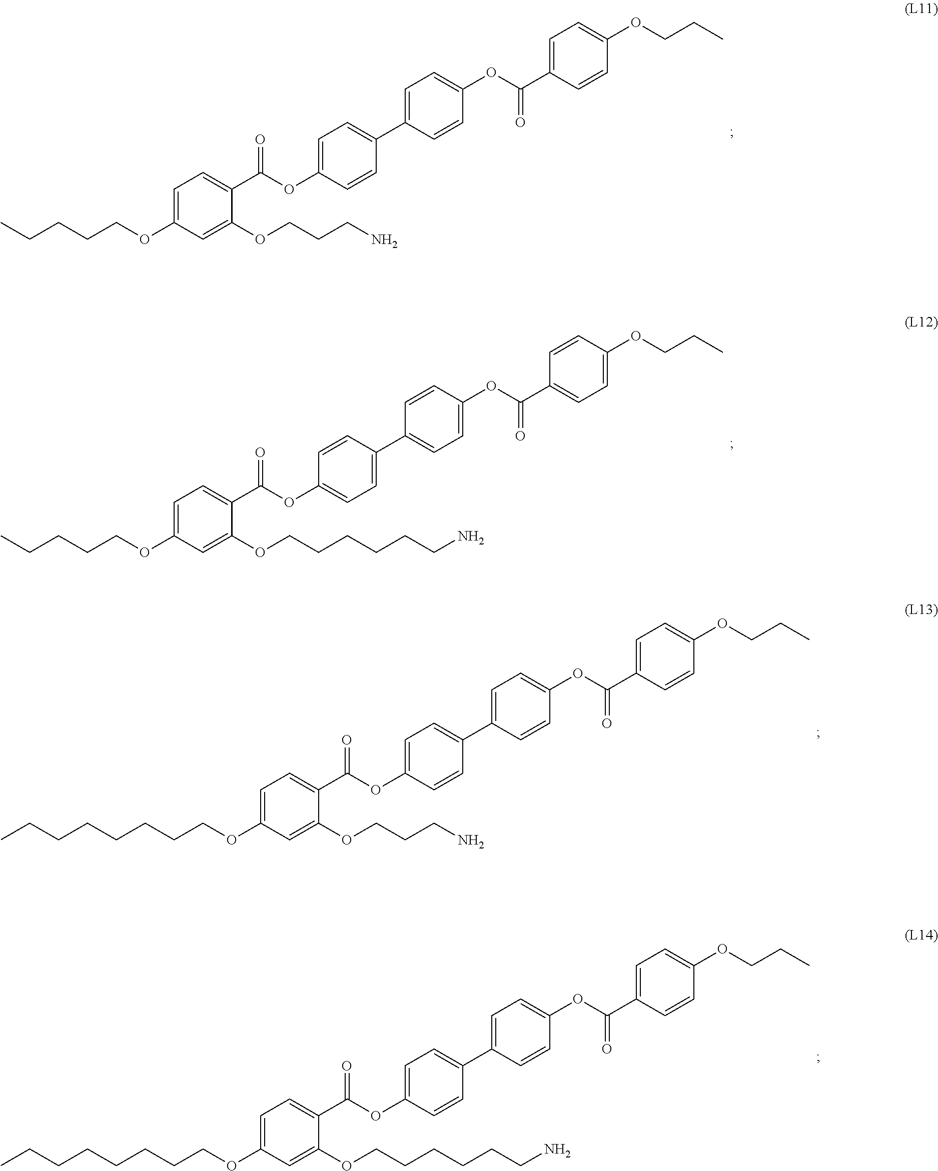

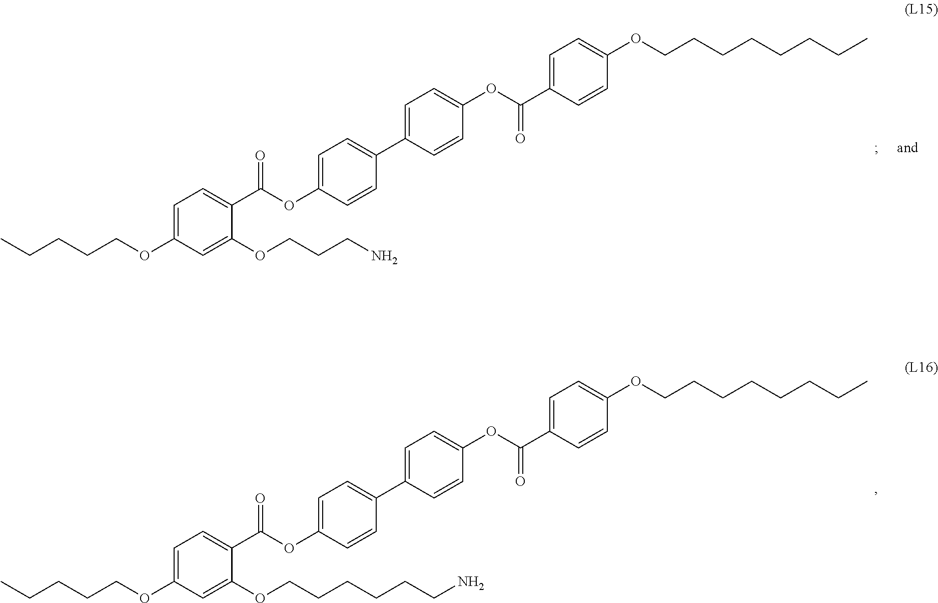

or combinations thereof.
Clause 30. The method of any one of clauses 27 to 29, wherein the power source is below the American National Standards Institute (ANSI) maximum permissible exposure limit.
Clause 31. The method of any one of clauses 27 to 30, wherein the maximum permissible exposure power density of the power source is 100 mW/cm\hat{ }2 or less.
Clause 32. The method of any one of clauses 27 to 31, wherein the maximum permissible exposure time to the power source is 6 minutes or less.
Clause 33. The method of any one of clauses 27 to 32, wherein the release of the substrate is activated through localized surface plasmon resonance (LSPR) stimuli.
Clause 34. The method of any one of clauses 27 to 33, wherein the release of the substrate is activated at an excitation wavelength of from 200 nm to 1 mm.
Clause 35. The method of any one of clauses 27 to 33, wherein the release of the substrate is activated at an excitation wavelength of from 400 nm to 1 mm.
Clause 36. The method of any one of clauses 27 to 35, wherein the release of the substrate is activated with a light that has a wavelength from 200 nm to 1 mm at a power density of 100 mW/cm\hat{ }2 or less.
Clause 37. The method of any one of clauses 27 to 36, wherein full release of the substrate is obtained in 6 minutes or less from the time of activation with a power source.
Clause 38. The method of any one of clauses 27 to 37, wherein the temperature change at the microcapsule surface upon activation with a power source is 50° C. or less.
Clause 39. The method of any one of clauses 27 to 38, wherein one or more substrates is an active agent.
Clause 40. The method of any one of clauses 27 to 39, wherein one or more substrates is live cells.
Clause 41. A kit for delivering one or more substrates to an individual, the kit comprising:

one or more containers comprising the self-assembled microcapsule of any one of clauses 1 to 24, wherein the substrate is selected from an active agent or live cells.

That which is claimed is:

1. A self-assembled microcapsule comprising organic ligand-functionalized nanoparticles and one or more substrates encapsulated inside the microcapsule, wherein the microcapsule releases the substrate upon activation with a power source and the maximum temperature change at the microcapsule surface upon activation with the power source is 75° C. or less.

2. The self-assembled microcapsule of claim 1, wherein the organic ligand has the structure of formula (I): 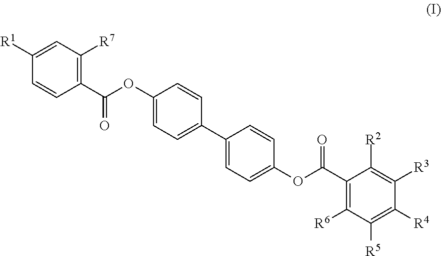
wherein
R¹ and R⁷ are each independently selected from, C₁-C₈ alkoxy, and C₁-C₈ alkoxy substituted with an amine or thiol group; and
R², R³, R⁴, R⁵ and R⁶ are each independently selected from H, halogen, hydroxyl, azido, alkyl, substituted alkyl, alkenyl, substituted alkenyl, alkynyl, substituted alkynyl, C₁-C₁₂ alkoxy, substituted alkoxy, amino, substituted amino, cycloalkyl, substituted cycloalkyl, heterocycloalkyl, substituted heterocycloalkyl, aryl, substituted aryl, heteroaryl, substituted heteroaryl, phosphate, substituted phosphate, phosphoryl, substituted phosphoryl, thiol and substituted thiol and combinations thereof.

3. The self-assembled microcapsule of claim 1 or 2, wherein the mean inter-particle separation of the nanoparticles is from 1 nm to 100 nm.

4. The self-assembled microcapsule of any one of claims 1 to 3, wherein the nanoparticles are composed of upconversion nanoparticles, plasmonic nanoparticles, or combinations thereof.

5. The self-assembled microcapsule of any one of claims 1 to 4, wherein the nanoparticles are composed of a material selected from a semiconductor material, a metal, a metal oxide, a metalloid, a metal coated material, an oxide, a magnetic material, a nanosome, a lipidsome and a polymer, or combinations thereof.

6. The self-assembled microcapsule of claim 5, wherein the nanoparticles are composed of gold nanoparticles, silver nanoparticles, zinc oxide nanoparticles, gold coated nanoparticles, silver coated nanoparticles, zinc coated nanoparticles or combinations thereof.

7. The self-assembled microcapsule of claim 5, wherein the nanoparticles are composed of iron oxide nanoparticles, cobalt nanoparticles, graphene coated iron oxide nanoparticles, graphene coated cobalt, silica coated iron oxide and silica coated cobalt or combinations thereof.

8. The self-assembled microcapsule of claim 6, wherein the nanoparticles are composed of gold nanoparticles.

9. The self-assembled microcapsule of any one of claims 1 to 8, wherein the microcapsule has a spherical surface.

10. The self-assembled microcapsule of any one of claims 1 to 9, wherein the organic ligand is selected from the group consisting of: 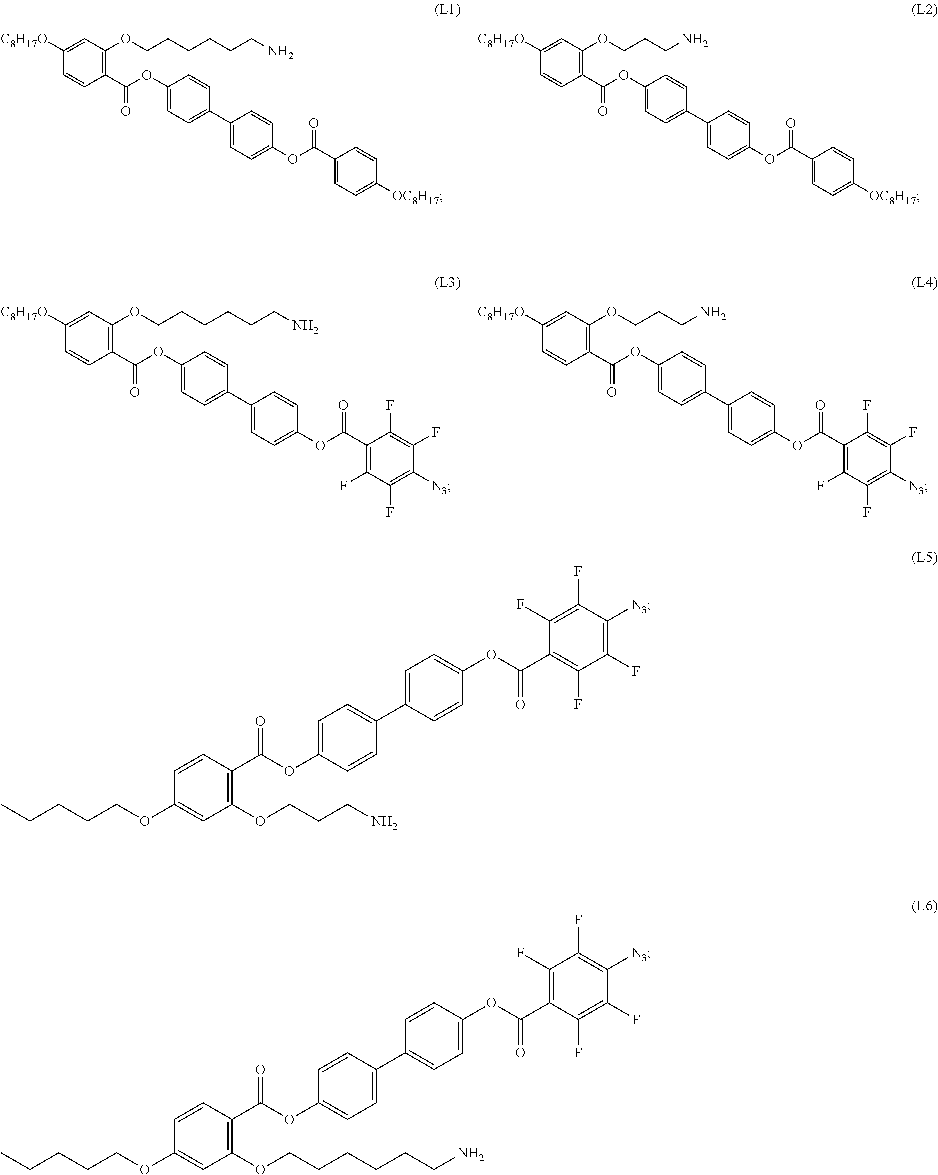 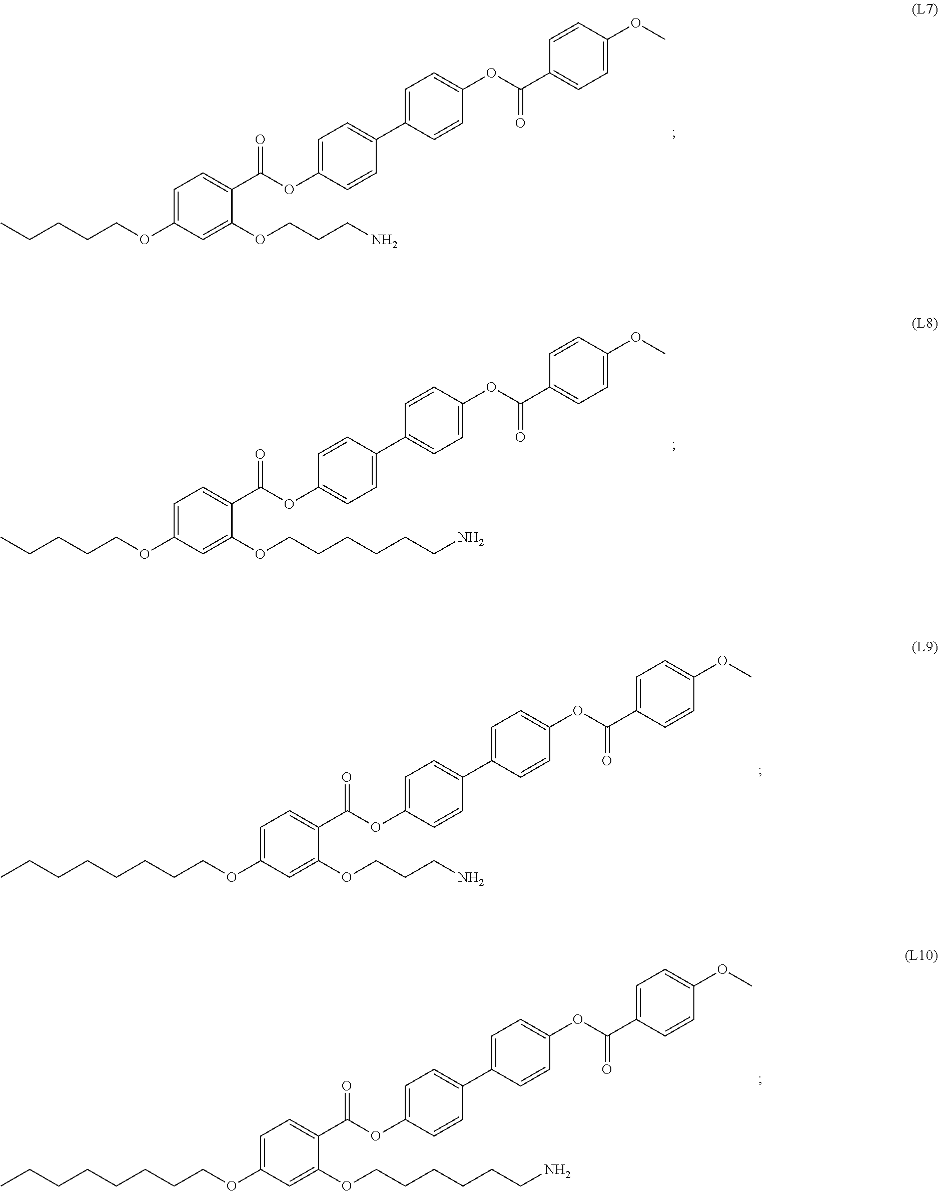 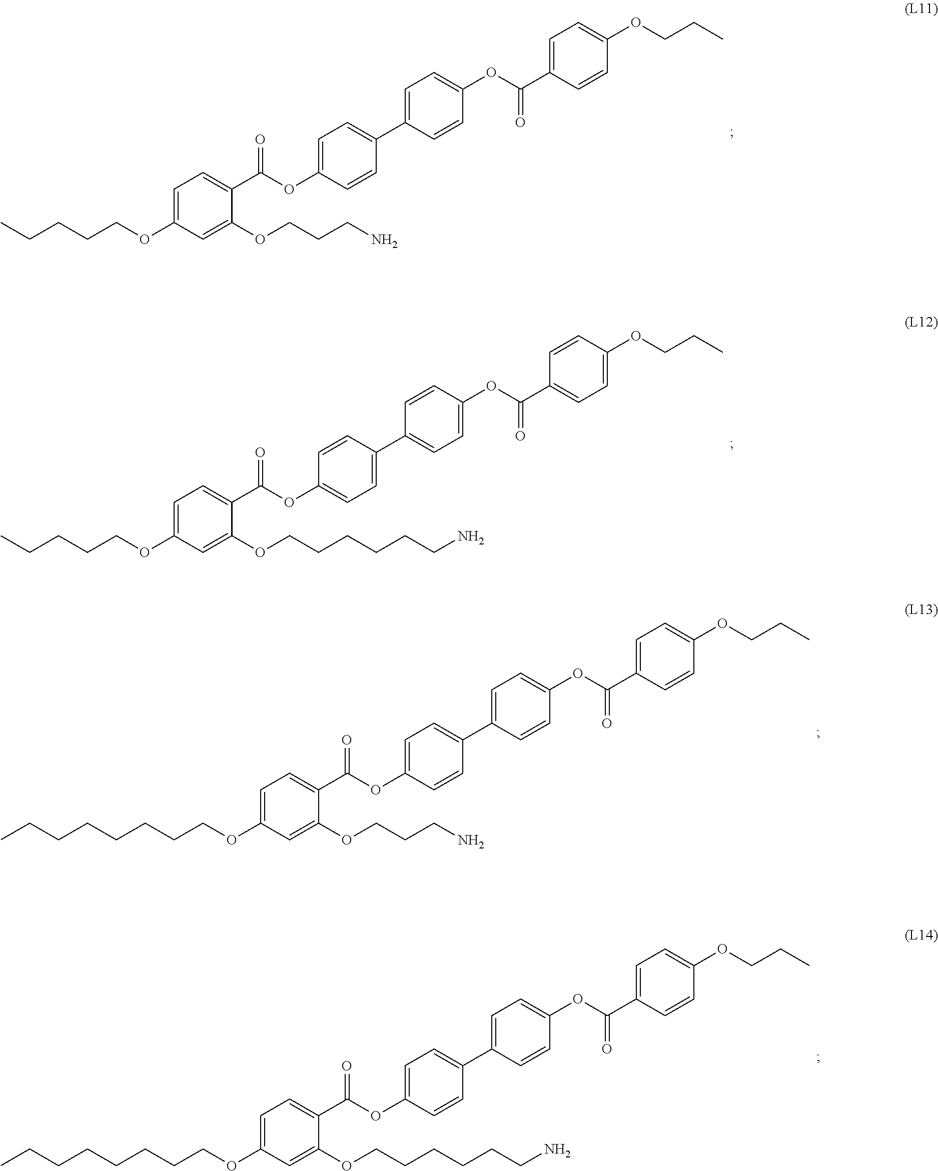 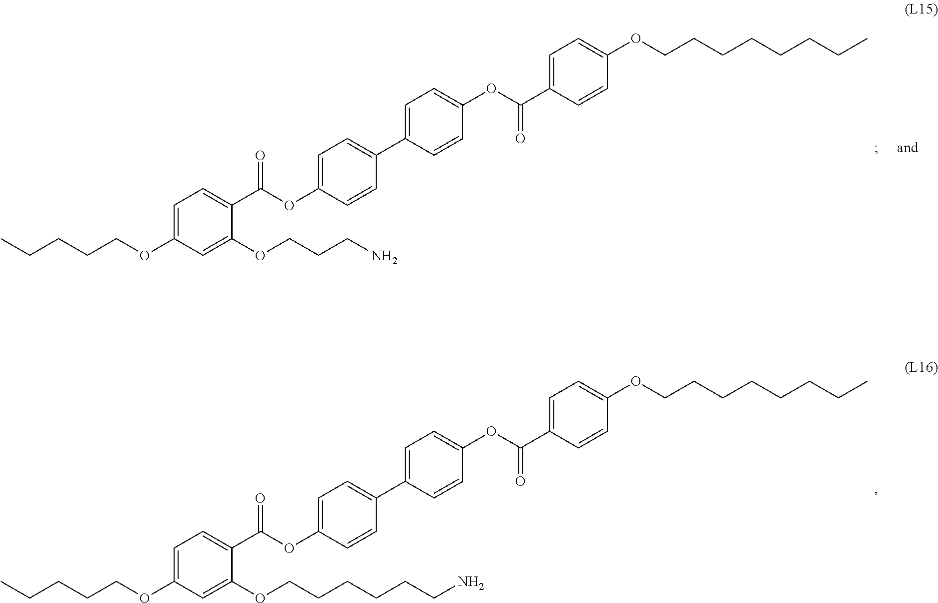
or combinations thereof.

11. The self-assembled microcapsule of claim 9, wherein the microcapsule has an average diameter of 100 nm to 100 μm.

12. The self-assembled microcapsule of claim 9, wherein the nanoparticles have an average diameter of 1 nm to 100 nm.

13. The self-assembled microcapsule of any one of claims 1 to 12, wherein the microcapsule has a thickness of from 1% to 50% of the volume of the microcapsule.

14. The self-assembled microcapsule of any one of claims 1 to 13, wherein the substrate is an active agent.

15. The self-assembled microcapsule of any one of claims 1 to 13, wherein the substrate is live cells.

16. The self-assembled microcapsule of any one of claims 1 to 15, wherein the power source is below the American National Standards Institute (ANSI) maximum permissible exposure limit.

17. The self-assembled microcapsule of any one of claims 1 to 16, wherein the maximum permissible exposure power density is 100 mW/cm\hat{ }2 or less.

18. The self-assembled microcapsule of any one of claims 1 to 17, wherein the maximum permissible exposure time to the power source is 6 minutes or less.

19. The self-assembled microcapsule of any one of claims 1 to 18, wherein the release of the substrate is activated through localized surface plasmon resonance (LSPR) stimuli.

20. The self-assembled microcapsule of any one of claims 1 to 19, wherein the release of the substrate is activated at an excitation wavelength of from 200 nm to 1 mm.

21. The self-assembled microcapsule of any one of claims 1 to 19, wherein the release of the substrate is activated at an excitation wavelength of from 400 nm to 1 mm.

22. The self-assembled microcapsule of any one of claims 1 to 21, wherein the release of the substrate is activated with a light that has a wavelength from 200 nm to 1 mm at a power density 100 mW/cm\hat{ }2 or less.

23. The self-assembled microcapsule of any one of claims 1 to 22, wherein full release of the substrate is obtained in 6 minutes or less from the time of activation with a power source.

24. The self-assembled microcapsule of any one of claims 1 to 23, wherein the temperature change at the microcapsule surface upon activation with a power source is 50° C. or less.

25. A composition comprising:
a liquid; and
a self-assembled microcapsule of any one of claims 1 to 24 in the liquid.

26. The composition of claim 25, wherein the liquid is a pharmaceutically acceptable liquid or a mesomorphic material.

27. A method of delivering one or more substrates to an individual, the method comprising:
administering an effective amount of a self-assembled microcapsule comprising, organic ligand-functionalized nanoparticles and one or more substrates encapsulated inside the microcapsule, to an individual; and
applying activation from a power source to release the one or more substrates, wherein the maximum temperature change at the microcapsule surface upon activation with the power source is 75° C. or less.

28. The method of claim 27, wherein the organic ligand has the structure of formula (I): 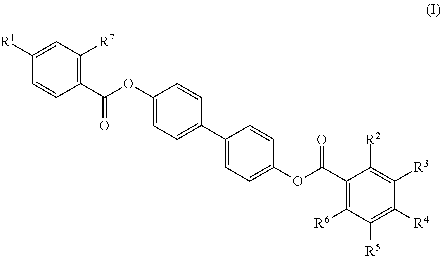
wherein
R¹ and R⁷ are each independently selected from, C₁-C₈ alkoxy, and C₁-C₈ alkoxy substituted with an amine or thiol group; and
R², R³, R⁴, R⁵ and R⁶ are each independently selected from H, halogen, hydroxyl, azido, alkyl, substituted alkyl, alkenyl, substituted alkenyl, alkynyl, substituted alkynyl, C₁-C₁₂ alkoxy, substituted alkoxy, amino, substituted amino, cycloalkyl, substituted cycloalkyl, heterocycloalkyl, substituted heterocycloalkyl, aryl, substituted aryl, heteroaryl, substituted heteroaryl, phosphate, substituted phosphate, phosphoryl, substituted phosphoryl, thiol and substituted thiol and combinations thereof.

29. The method of claim 27 or 28, wherein the organic ligand is selected from the group consisting of: 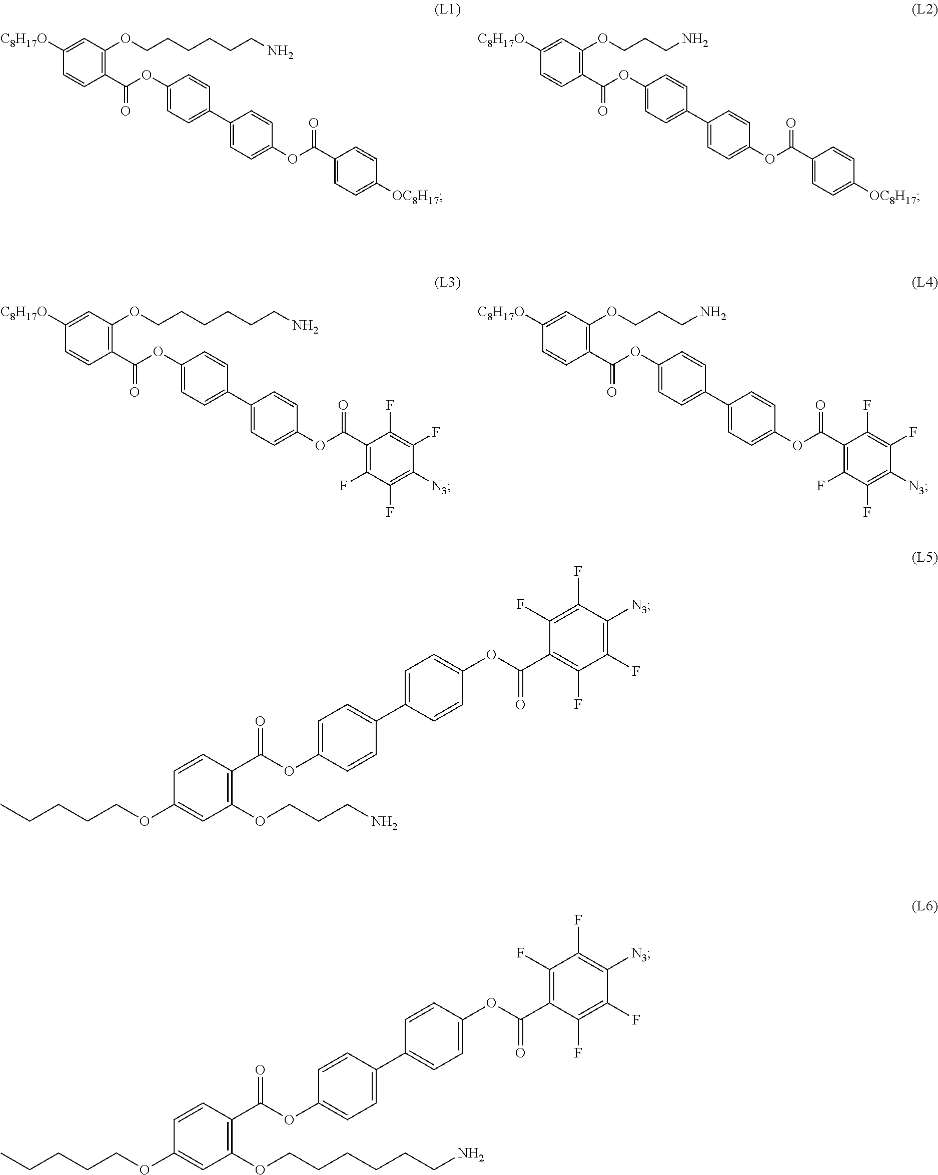 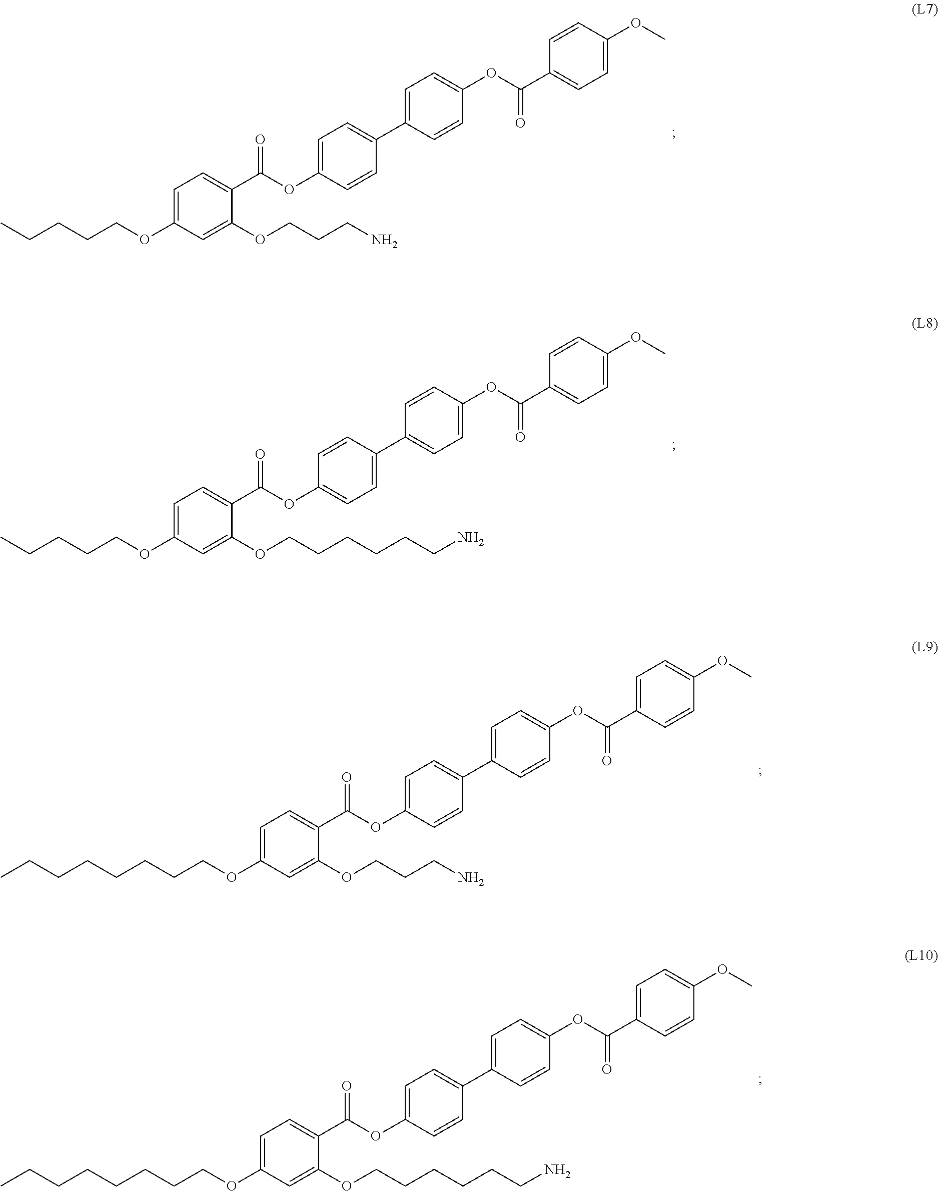 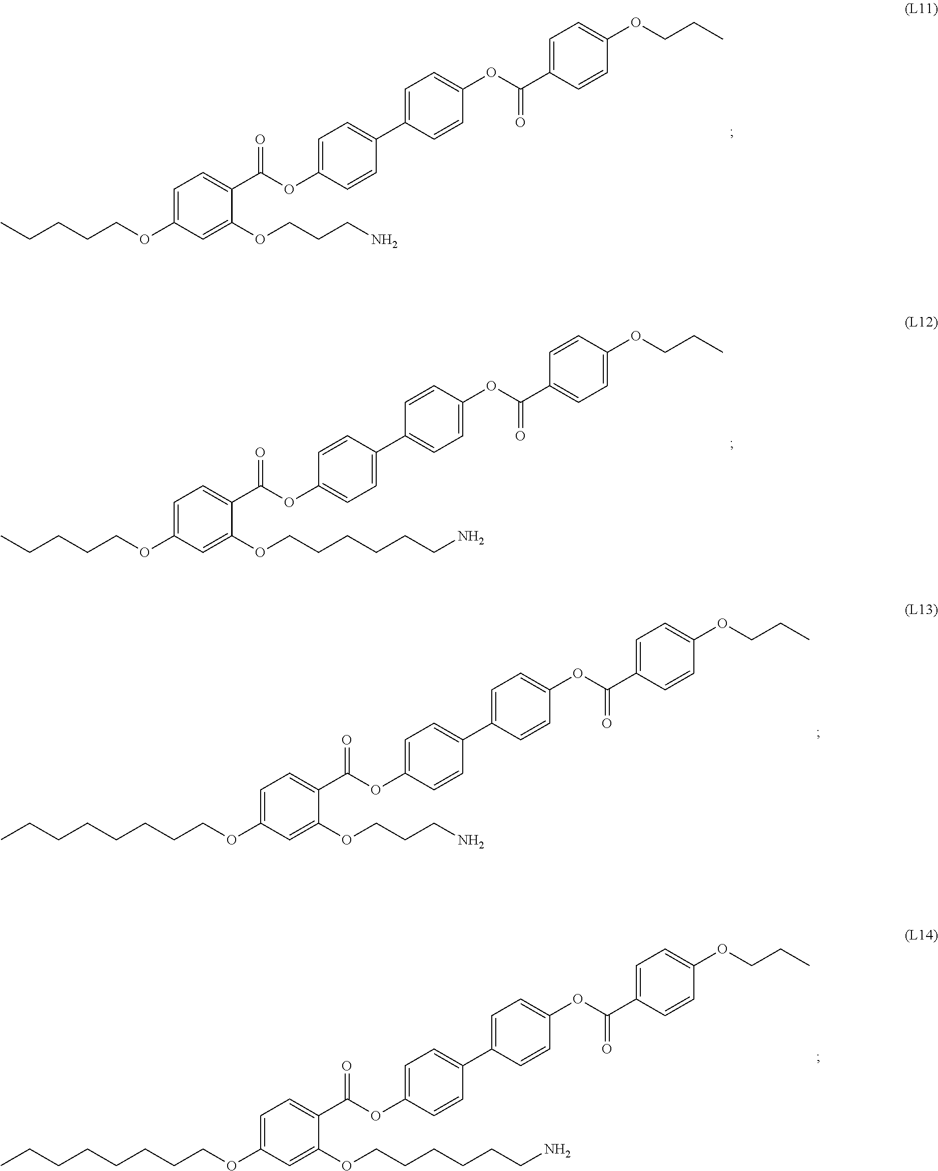 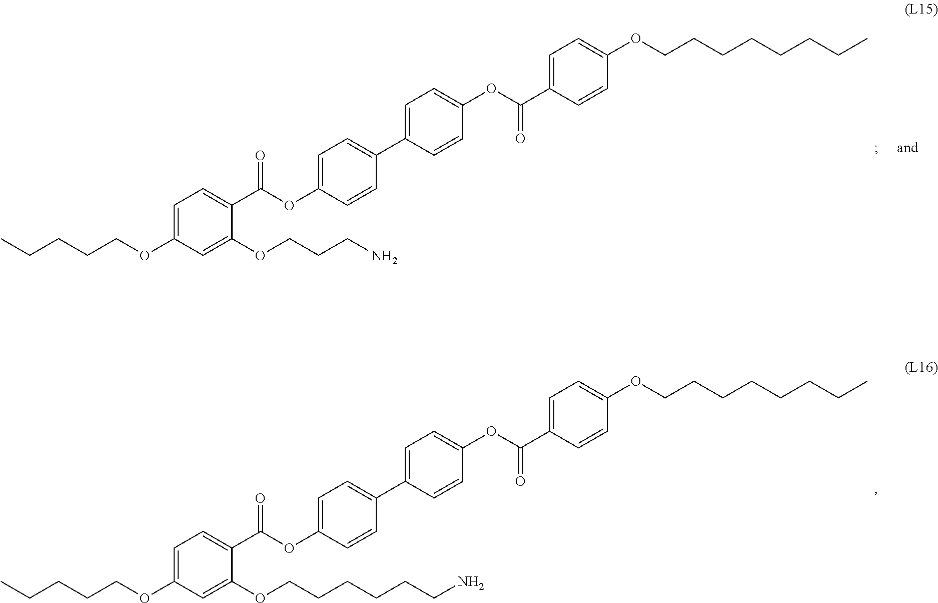
or combinations thereof.

30. The method of any one of claims 27 to 29, wherein the power source is below the American National Standards Institute (ANSI) maximum permissible exposure limit.

31. The method of any one of claims 27 to 30, wherein the maximum permissible exposure power density of the power source is 100 mW/cm\hat{ }2 or less.

32. The method of any one of claims 27 to 31, wherein the maximum permissible exposure time to the power source is 6 minutes or less.

33. The method of any one of claims 27 to 32, wherein the release of the substrate is activated through localized surface plasmon resonance (LSPR) stimuli.

34. The method of any one of claims 27 to 33, wherein the release of the substrate is activated at an excitation wavelength of from 200 nm to 1 mm.

35. The method of any one of claims 27 to 33, wherein the release of the substrate is activated at an excitation wavelength of from 400 nm to 1 mm.

36. The method of any one of claims 27 to 35, wherein the release of the substrate is activated with a light that has a wavelength from 200 nm to 1 mm at a power density of 100 mW/cm\hat{ }2 or less.

37. The method of any one of claims 27 to 36, wherein full release of the substrate is obtained in 6 minutes or less from the time of activation with a power source.

38. The method of any one of claims 27 to 37, wherein the temperature change at the microcapsule surface upon activation with a power source is 50° C. or less.

39. The method of any one of claims 27 to 38, wherein one or more substrates is an active agent.

40. The method of any one of claims 27 to 39, wherein one or more substrates is live cells.

41. A kit for delivering one or more substrates to an individual, the kit comprising:
one or more containers comprising the self-assembled microcapsule of any one of claims 1 to 24, wherein the substrate is selected from an active agent or live cells.
